(19) United States
(12) Patent Application Publication
(10) Pub. No.: US 2023/0255502 A1
(43) Pub. Date: Aug. 17, 2023

(54) DISPLAY DEVICE AND A METHOD FOR MEASURING BLOOD PRESSURE USING THE SAME

(71) Applicant: SAMSUNG DISPLAY CO., LTD., YONGIN-SI (KR)

(72) Inventors: Jong Yeop AN, Hwaseong-si (KR); Gyeong Ub MOON, Suwon-si (KR); Hyeon Jun LEE, Hanam-si (KR); Bo Ram CHOI, Asan-si (KR)

(21) Appl. No.: 17/938,424

(22) Filed: Oct. 6, 2022

(30) Foreign Application Priority Data
Feb. 16, 2022 (KR) 10-2022-0020291

(51) Int. Cl. A61B 5/021; A61B 5/00

(52) U.S. Cl. CPC A61B 5/02116; A61B 5/0082; A61B 5/743; A61B 5/7425

(57) ABSTRACT

A display device is provided. The display device includes a display panel including a pixel configured to display an image and a photo-sensor configured to sense incident light, a pressure sensor disposed on one surface of the display panel and configured to sense a pressure applied via a portion of a user’s body, and a processor. The processor is configured to generate a pulse wave signal according to an amount of incident light sensed by the photo-sensor and an optical signal corresponding to the amount of incident light in response to determining that a pressure measurement value corresponding to the pressure is within a pressure request range corresponding to a pressure interval, and calculate blood pressure information based on the pulse wave signal.

### CROSS-REFERENCE TO RELATED APPLICATION(S)

This application claims priority under 35 U.S.C. § 119 to Korean Patent Application No. 10-2022-0020291, filed on Feb. 16, 2022 in the Korean Intellectual Property Office, the disclosure of which is incorporated by reference herein in its entirety.

### TECHNICAL FIELD

The inventive concept relates generally to a display device, and more specifically to a display device and a method for measuring blood pressure using the same.

### DISCUSSION OF RELATED ART

A display device is an output device for presentation of information in a visual or tactile form. For example, display devices may be implemented in or as televisions (TVs), monitors, portable smartphones, tablet personal computers (PCs), and the like. A display device may be provided to perform additional functions, such as a camera function, a fingerprint sensor function, and the like.

A display device may be used in the healthcare context. For example, a display device may be used to acquire biometric information pertaining to a user’s health.

### SUMMARY

Aspects of the present disclosure provide a display device capable of detecting a user’s blood pressure, thereby avoiding the inconvenience of measuring the blood pressure using conventional oscillometric pulse measurement devices.

Aspects of the disclosure provide a display device capable of separately re-measuring only a pulse wave signal corresponding to a pressure interval for which a pulse wave signal might not be accurately analyzed, and a blood pressure measurement method using the same.

Aspects of the disclosure also provide a display device capable of displaying a pulse wave signal measurement section, a pulse wave signal regeneration section, and a pressure measurement value on a display panel in real time, and a blood pressure measurement method using the same.

According to at least one embodiment, a display device for measuring a blood pressure is provided. In some embodiments, the display device includes a display panel including a pixel configured to display an image and a photo-sensor configured to sense incident light, a pressure sensor disposed on one surface of the display panel and configured to sense a pressure applied via a portion of a user’s body, and a processor. The processor is configured to generate a pulse wave signal according to an amount of incident light sensed by the photo-sensor and an optical signal corresponding to the amount of incident light in response to determining that a pressure measurement value corresponding to the pressure is within a pressure request range corresponding to a pressure interval, and calculate blood pressure information based on the pulse wave signal.

In some embodiments, the processor is further configured to analyze the pressure interval as a regeneration section, and regenerate the pulse wave signal according to an amount of light sensed by the photo-sensor corresponding to the pressure interval and the optical signal corresponding to the amount of incident light.

In some embodiments, the processor is further configured to generate a peak detection signal using peak values of the pulse wave signal, and determine the pressure interval as the regeneration section when a number of peak values of the peak detection signal that exceed at least one threshold is two or more.

In some embodiments, a first area of the display panel displays an image of values of the pulse wave signal generated corresponding to a plurality of pressure intervals via a first user interface, and displays an image of the regeneration section via the first user interface.

In some embodiments, a first area of the display panel is configured to display an image of values of the pulse wave signal generated corresponding to a plurality of pressure intervals via a first user interface, and display an image of the regeneration section via the first user interface.

In some embodiments, a first area of the display panel is configured to display a first image of values of the pulse wave signal generated based on the pressure interval via a first user interface, and a second area of the display panel is configured to display a second image of the pressure request range and the pressure measurement value via a second user interface.

In some embodiments, the pressure request range includes a first requested pressure and a second requested pressure higher than the first requested pressure, and the second image further includes the first requested pressure and the second requested pressure.

In some embodiments, first to N-th pressure request ranges respectively correspond to first to N-th pressure intervals, and bounding values of a succeeding pressure request range are greater than bounding values of a preceding pressure request range.

In some embodiments, the processor is further configured to generate a peak detection signal using a peak value of the pulse wave signal, calculate a pressure value corresponding to the peak value, and calculate a diastolic blood pressure, a systolic blood pressure, and a mean blood pressure according to the pressure value.

In some embodiments, the processor is further configured to calculate the diastolic blood pressure as being equal to a value in a range of about 60% to about 80% of the pressure value, and calculate the systolic blood pressure as being equal to a value in a range of about 120% to about 140% of the pressure value.

In some embodiments, a greatest amplitude in a cycle of the pulse wave signal is a pulse wave maximum value, a second greatest amplitude in the cycle of the pulse wave signal is a reflected pulse wave value, and the processor is further configured to calculate a reflected pulse wave ratio as a ratio of the reflected pulse wave value to the pulse wave maximum value.

In some embodiments, the reflected pulse wave ratio includes a first period in which the reflected pulse wave ratio fluctuates within a first range, a second period in which the reflected pulse wave ratio fluctuates within a second range, and a third period in which the reflected pulse wave ratio fluctuates within a third range, and a width of the first range and a width of the third range are smaller than a width of the second range.

In some embodiments, the processor is further configured to analyze the reflected pulse wave ratio to detect a start point in time of the second period, calculate a third pressure value corresponding to the pulse wave signal at the start point in time of the second period, determine a diastolic blood pressure as the third pressure value, calculate a fourth pressure value corresponding to the pulse wave signal at a start point in time of the third period after the second period, and determine a systolic blood pressure as the fourth pressure value.

In some embodiments, the reflected pulse wave ratio is equal to or greater than one, and the processor is further configured to regenerate the pulse wave signal according to a second amount of light sensed by the photo-sensor and the optical signal corresponding to the second amount of light.

According to at least one embodiment, a method for measuring a blood pressure is provided. In some embodiments, the method includes sensing, via a pressure sensor of a display device, a pressure applied via a portion of a user’s body, generating a pulse wave signal according to an amount of light sensed by a photo-sensor of a display device and an optical signal corresponding to the amount of light in response to determining that a pressure measurement value corresponding to the pressure is within a pressure request range corresponding to a pressure interval, regenerating the pulse wave signal according to a second amount of light sensed by the photo-sensor and the optical signal corresponding to the second amount of light in response to analyzing the pressure interval as a regeneration pressure interval, and calculating blood pressure information based on the regenerated pulse wave signal.

In some embodiments, the method further includes generating a peak detection signal using peak values of the pulse wave signal, and determining the pressure interval as the regeneration pressure interval when the number of peak values of the peak detection signal that exceed a threshold is two or more.

In some embodiments, the method further includes displaying an image of the pulse wave signal via a first user interface, and displaying an image of the pressure measurement value and a pressure request range corresponding to the pressure measurement value via a second user interface.

In some embodiments, the method further includes displaying an image of the regeneration pressure interval via the first user interface.

In some embodiments, a greatest amplitude in a cycle of the pulse wave signal is a pulse wave maximum value, a second greatest amplitude in the cycle of the pulse wave signal is a reflected pulse wave value, and the method further includes calculating a reflected pulse wave ratio as the ratio of the reflected pulse wave value to the pulse wave maximum value.

In some embodiments, the method further includes determining that the pulse wave ratio is equal to or greater than 1, wherein the pulse wave signal is regenerated based on the determination.

In some embodiments, the method further includes generating a peak detection signal using peak values of the pulse wave signal, calculating a pressure value corresponding to the peak value of the peak detection signal, and calculating a diastolic blood pressure, a systolic blood pressure, and a mean blood pressure according to the pressure value.

According to at least one embodiment, a method for measuring a blood pressure is provided. In some embodiments, the method includes displaying a pressure request range via a user interface, receiving a pressure within the pressure request range via a portion of a user’s body, generating a pulse wave signal based on the pressure and a first amount of light reflected from the user, determining that two or more values of the pulse wave signal exceed a threshold, regenerating the pulse wave signal to obtain a regenerated pulse wave signal and a second amount of light reflected from the user, and calculating the blood pressure of the user based on the regenerated pulse wave signal.

Therefore, according to some embodiments, a display device may measure a blood pressure of a user by sensing light reflected from a blood vessel or the like of a finger of the user by a photo-sensor of a display panel and analyzing a pulse wave signal according to an amount of the sensed light.

For example, in some embodiments, a pulse wave signal generated according to a pressure interval might not be analyzed due to the pulse wave signal being unstable or irregular, and the pulse wave signal is therefore separately re-measured and regenerated. Accordingly, by only regenerating values of the pulse wave signal that are determined to be unstable or irregular, an efficiency and accuracy of blood pressure measurement may be increased.

In another example, in some embodiments, an efficiency and accuracy of a blood pressure measurement may be increased by displaying a pressure measurement value for measuring the pulse wave signal, a pulse wave signal measurement section, and a pulse wave signal re-measurement and/or regeneration section on a display panel in real time.

### BRIEF DESCRIPTION OF THE DRAWINGS

The above and other aspects and features of the disclosure will become more apparent by describing in detail embodiments thereof with reference to the attached drawings, in which:

FIG. 1 is a plan view of a display device according to at least one embodiment;

FIG. 2 is a block diagram illustrating the display device of FIG. 1 according to at least one embodiment;

FIG. 3 is a plan layout view of pixels and photo-sensors of a display cell according to at least one embodiment;

FIG. 4 is a cross-sectional view taken along line I-I′ of FIG. 3;

FIG. 5 is a flowchart illustrating a process for blood pressure measurement according to at least one embodiment;

FIG. 6 is a plan view illustrating a user interface of the display device of FIG. 1 according to at least one embodiment;

FIG. 7 is an enlarged plan view of a first user interface of FIG. 6;

FIG. 8 is a flowchart illustrating a process for generating a pulse wave signal according to at least one embodiment;

FIGS. 9 and 10 are enlarged plan views of a second user interface of the display device of FIG. 1 according to at least one embodiment;

FIG. 11 is a graph illustrating a pressure measurement value according to a pressure applying time;

FIG. 12 is a graph illustrating a pulse wave signal according to a pressure applying time;

FIG. 13 is a graph illustrating a relationship between a pressure and a pulse wave signal;

FIG. 14 is a plan view illustrating a user interface of the display device of FIG. 1 according to at least one embodiment;

FIG. 15 is a flowchart illustrating a process for regenerating a pulse wave signal according to at least one embodiment;

FIGS. 16 and 17 are graphs illustrating pulse wave signals according to at least one embodiment;

FIG. 18 is a plan view illustrating a user interface of the display device of FIG. 1 according to at least one embodiment;

FIG. 19 is an enlarged plan view of a first user interface of FIG. 18;

FIG. 20 is a flowchart illustrating another a process for regenerating a pulse wave signal according to at least one embodiment;

FIGS. 21 to 23 are enlarged graphs of waveforms of the pulse wave signal illustrated in FIG. 16;

FIG. 24 is a flowchart illustrating a process for calculating a blood pressure of a user using a generated pulse wave signal according to at least one embodiment;

FIG. 25 is a flowchart illustrating a process for calculating a blood pressure using a generated pulse wave signal and a reflected pulse wave ratio according to at least one embodiment;

FIG. 26 is a graph illustrating a pulse wave signal according to at least one embodiment;

FIG. 27 is a plan view illustrating a user interface according to at least one embodiment;

FIGS. 28 and 29 are plan views illustrating user interfaces according to at least one embodiment;

FIG. 30 is a flowchart illustrating a process for blood pressure measurement using a display device according to at least one embodiment;

FIG. 31 is a graph illustrating a pulse wave signal according to at least one embodiment;

FIG. 32 is a flowchart illustrating a blood pressure measurement process using a display device according to at least one embodiment; and

FIGS. 33 to 35 are graphs illustrating pulse wave signals according to at least one embodiment.

### DETAILED DESCRIPTION

Embodiments of the inventive concept will be described more fully hereinafter with reference to the accompanying drawings. Like reference numerals may refer to like elements throughout the accompanying drawings.

It will be understood that when a component, such as a film, a region, a layer, or an element, is referred to as being “on”, “connected to”, “coupled to”, or “adjacent to” another component, it can be directly on, connected, coupled, or adjacent to the other component, or intervening components may be present. It will also be understood that when a component is referred to as being “between” two components, it can be the only component between the two components, or one or more intervening components may also be present. It will also be understood that when a component is referred to as “covering” another component, it can be the only component covering the other component, or one or more intervening components may also be covering the other component. Other words use to describe the relationship between elements should be interpreted in a like fashion.

It will be understood that, although the terms “first,” “second,” etc. may be used herein to describe various elements, these elements should not be limited by these terms. These terms are only used to distinguish one element from another element. For instance, a first element discussed below could be termed a second element without departing from the teachings of the present invention. Similarly, the second element could also be termed the first element.

As used herein, the singular forms “a,” “an” and “the” are intended to include the plural forms as well, unless the context clearly indicates otherwise.

Herein, when one value is described as being about equal to another value or being substantially the same as or equal to another value, it is to be understood that the values are equal to each other to within a measurement error, or if measurably unequal, are close enough in value to be functionally equal to each other as would be understood by a person having ordinary skill in the art. For example, the term “about” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” may mean within one or more standard deviations as understood by one of the ordinary skill in the art. Further, it is to be understood that while parameters may be described herein as having “about” a certain value, according to some embodiments, the parameter may be exactly the certain value or approximately the certain value within a measurement error as would be understood by a person having ordinary skill in the art.

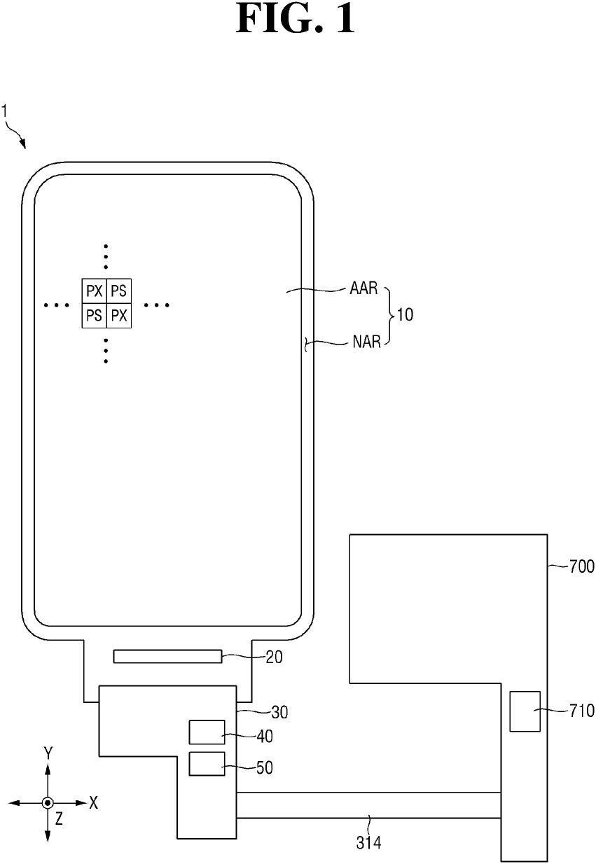

FIG. 1 is a plan view of a display device according to at least one embodiment.

In FIG. 1, a first direction X, a second direction Y, and a third direction Z are indicated. According to some aspects, the first direction X is a direction parallel to a first side of a display device 1. According to some aspects, the first direction X is a transverse direction of the display device 1. According to some aspects, the second direction Y is a direction parallel to a second side of the display device 1 that contacts the first side of the display device 1. According to some aspects, the second direction Y is a longitudinal direction of the display device 1. In some embodiments, the second direction Y is orthogonal to the first direction X. Hereinafter, for convenience of explanation, one side in the first direction X may refer to a right direction, the other side in the first direction X may refer to a left direction, one side in the second direction Y may refer to an upper direction, and the other side in the second direction Y may refer to a lower direction. According to some aspects, a third direction Z is a thickness direction of the display device 1. According to some aspects, the third direction Z is orthogonal to the second direction Y and the first direction X.

Unless noted otherwise, the terms “upper”, “upper surface”, and “upper side” are expressed with respect to the third direction Z and refer to a display surface side with respect to a display panel 10, and the terms “lower”, “lower surface”, and “lower side” are expressed with respect to the third direction Z and refer to an opposite side to a display surface with respect to the display panel 10.

Referring to FIG. 1, the display device 1 may be implanted as or included in various electronic devices that include a display screen. According to some aspects, the display device 1 may be implemented in or as a mobile phone, a smartphone, a tablet personal computer (PC), a mobile communication terminal, an electronic notebook, an electronic book, a personal digital assistant (PDA), a portable multimedia players (PMP), a navigation device, an ultra-mobile PC (UMPC), a television, a game machine, a wrist watch-type electronic device, a head-mounted display, a monitor of a personal computer, a laptop computer, a vehicle instrument board, a digital camera, a camcorder, an external billboard, an electric sign, various medical devices, various inspection devices, various home appliances including a display area, such as a refrigerator or a washing machine, an Internet of Things (IoT) device, or the like. In some embodiments, display device 1 is a smartphone, a tablet PC, a laptop computer, or the like.

According to some aspects, the display device 1 includes a display panel 10, a panel driving circuit 20, a circuit board 30, a pulse wave sensing circuit 40, a pressure sensing circuit 50, a main circuit board 700, and a processor 710.

According to some aspects, the display panel 10 includes an active area AAR and a non-active area NAR. According to some aspects, the active area AAR includes a display area that displays a screen. According to some aspects, the active area AAR at least partially overlaps the display area. In some embodiments, the active area AAR completely overlaps the display area. In some embodiments, a plurality of pixels PX displaying an image is disposed in the display area. In some embodiments, each pixel PX includes a light-emitting unit that emits light. In some embodiments, the light-emitting unit is a diode.

According to some aspects, the active area AAR includes a light-sensing area. In some embodiments, the light-sensing area is an area that responds to light and is configured to sense an amount, a wavelength, or other characteristic of incident light. In some embodiments, the light-sensing area at least partially overlaps the display area. In at least one embodiment, the light-sensing area completely overlaps the active area AAR. In this case, the light-sensing area and the display area may be the same as each other. In another embodiment, the light-sensing area is disposed in a portion of the active area AAR and is omitted from the remaining portions of the active area AAR. For example, in at least one embodiment, the light sensing-area is disposed in a portion of the active area AAR that is used for fingerprint recognition and is omitted from a portion of the active area AAR that is not used for fingerprint recognition. In this case, the light-sensing area may overlap a portion of the display area and might not overlap another portion of the display area.

According to some aspects, a plurality of photo-sensors PS responding to light are disposed in the light-sensing area. According to some aspects, the non-active area NAR is disposed around the active area AAR. In an example, the non-active area NAR at least partially surrounds the active area AAR. According to some aspects, the panel driving circuit 20 is disposed in the non-active area NAR. According to some aspects, the panel driving circuit 20 drives the plurality of pixels PX and/or the plurality of photo-sensors PS. According to some aspects, the panel driving circuit 20 outputs signals and voltages for driving the display panel 10. According to some aspects, the panel driving circuit 20 is formed as an integrated circuit (IC) and is mounted on the display panel 10. According to some aspects, signal lines for transferring signals between the panel driving circuit 20 and the active area AAR are disposed in the non-active area NAR. In at least one embodiment, the panel driving circuit 20 is mounted on the circuit board 30.

According to some aspects, the circuit board 30 is attached to the display panel 10 via an anisotropic conductive film (ACF). According to some aspects, lead lines of the circuit board 30 are electrically connected to pad parts of the display panel 10. According to some aspects, the circuit board 30 is a flexible printed circuit board. According to some aspects, the circuit board 30 is a flexible film such as a chip on film.

According to some aspects, the pulse wave sensing circuit 40 is disposed on the circuit board 30. According to some aspects, the pulse wave sensing circuit 40 is formed as an integrated circuit and is attached to an upper surface of the circuit board 30. According to some aspects, the pulse wave sensing circuit 40 is connected to a display layer of the display panel 10. According to some aspects, the pulse wave sensing circuit 40 senses a photocurrent generated by photocharges incident on the plurality of photo-sensors PS of the display panel 10. According to some aspects, the pulse wave sensing circuit 40 recognizes a pulse wave of a user based on the photocurrent. In an example, in some embodiments, the pulse wave sensing circuit 40 recognizes a pulse wave reflected from a user based on the photocurrent.

According to some aspects, the pressure sensing circuit 50 is disposed on the circuit board 30. According to some aspects, the pressure sensing circuit 50 is formed as an integrated circuit and is attached to the upper surface of the circuit board 30. According to some aspects, the pressure sensing circuit 50 is connected to the display layer of the display panel 10. According to some aspects, the pressure sensing circuit 50 senses electrical signals via one or more pressures applied to one or more pressure sensors of the display panel 10. According to some aspects, the pressure sensing circuit 50 generates pressure data according to a change in the electrical signal sensed by the pressure sensor and transmits the pressure data to the processor 710.

According to some aspects, the main circuit board 700 is a printed circuit board or a flexible printed circuit board. According to some aspects, the main circuit board 700 includes the processor 710. According to some aspects, the processor 710 is an intelligent hardware device, such as a general-purpose processing component, a digital signal processor (DSP), a central processing unit (CPU), a graphics processing unit (GPU), a microcontroller, an application specific integrated circuit (ASIC), a field programmable gate array (FPGA), a programmable logic device, a discrete gate or transistor logic component, a discrete hardware component, or any combination thereof. In some cases, the processor 710 is configured to operate a memory array using a memory controller. In other cases, a memory controller is integrated into the processor 710. In some cases, the processor 710 is configured to execute computer-readable instructions stored in a memory unit of the display device 1 to perform various functions. In some embodiments, the processor 710 includes special purpose components for modem processing, baseband processing, digital signal processing, or transmission processing.

According to some aspects, the display device 1 includes a memory unit including one or more memory devices. Examples of a memory device include random access memory (RAM), read-only memory (ROM), or a hard disk. Examples of memory devices include solid state memory and a hard disk drive. In some examples, memory is used to store computer-readable, computer-executable software including instructions that, when executed, cause the processor 710 to perform various functions described herein. In some cases, the memory unit includes a basic input/output system (BIOS) that controls basic hardware or software operations, such as an interaction with peripheral components or devices. In some cases, the memory unit includes a memory controller that operates memory cells of the memory unit. For example, the memory controller may include a row decoder, column decoder, or both. In some cases, the memory cells within the memory unit store information in the form of a logical state.

According to some aspects, the processor 710 controls one or more functions of the display device 1. For example, in some embodiments, the processor 710 outputs digital video data to the panel driving circuit 20 through the circuit board 30, so that the display panel 10 displays an image. In an example, in some embodiments, the processor 710 receives touch data from a touch driving circuit, determines one or more touch coordinates of the user, and executes an application indicated by an icon displayed on a portion of the display panel 10 corresponding to the touch coordinates of the user.

According to some aspects, the processor 710 calculates a pulse wave signal PPG based on a change in blood pressure corresponding to a heartbeat according to an optical signal input from the pulse wave sensing circuit 40. According to some aspects, the processor 710 calculates a touch pressure of the user according to the electrical signal input from the pressure sensing circuit 50. According to some aspects, the processor 710 measures a blood pressure of the user based on the pulse wave signal PPG and a pressure provided by the user. According to some aspects, the processor 710 is an application processor formed of an integrated circuit, a central processing unit, a system chip, or a combination thereof.

According to some aspects, a mobile communication module capable of transmitting and receiving wireless signals to and from at least one of a base station, an external terminal, and a server over a mobile communication network is mounted on the main circuit board 700. According to some aspects, the wireless signal includes various types of data according to a transmission and/or a reception of a voice signal, a video call signal, or a text/multimedia message.

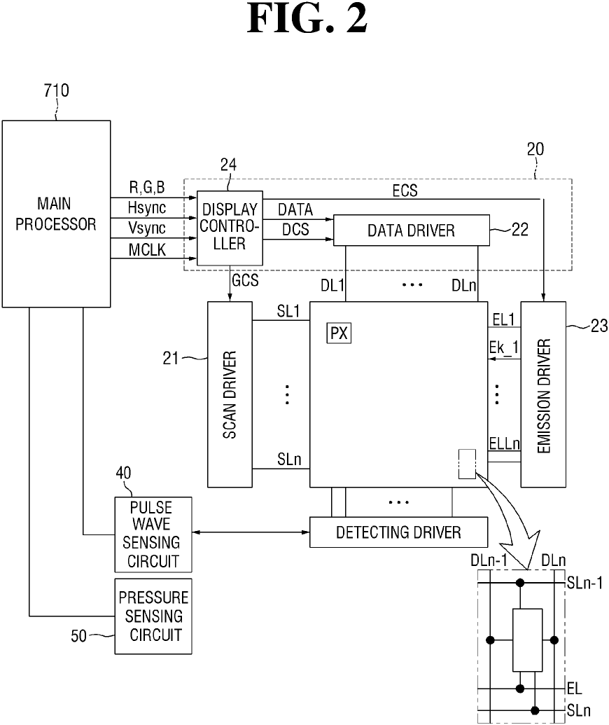

FIG. 2 is a block diagram illustrating the display device of FIG. 1 according to at least one embodiment. Referring to FIG. 2, according to some aspects, the display device 1 includes a display panel 10 including a plurality of pixels PX, a panel driving circuit 20, a scan driver 21, an emission driver 23, a pulse wave sensing circuit 40, a pressure sensing circuit 50, and a processor 710.

According to some aspects, the processor drives and controls the pulse wave sensing circuit 40, the pressure sensing circuit 50, and a display controller 24. According to some aspects, the processor 710 receives an optical signal from the pulse wave sensing circuit 40. According to some aspects, the processor 710 calculates a pulse wave signal PPG based on a change in blood pressure corresponding to a heartbeat according to the optical signal.

In some embodiments, the processor 710 receives an electrical signal from the pressure sensing circuit 50. In some embodiments, the processor 710 calculates a touch pressure of a user according to the electrical signal. In some embodiments, the processor 710 calculates a blood pressure of the user based on the pulse wave signal PPG and the pressure signal.

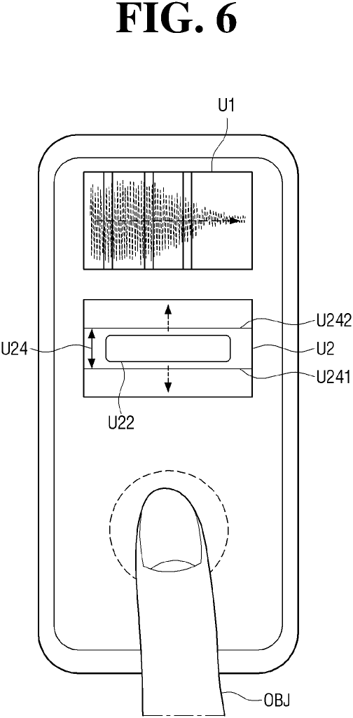

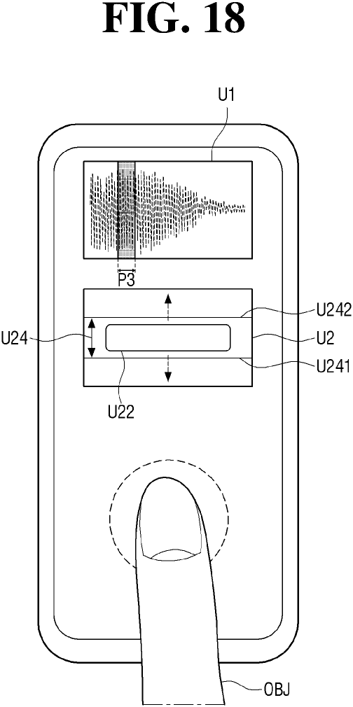

According to some aspects, the processor 710 outputs image information to the display controller 24. For example, in some embodiments, the processor 710 outputs image information including the calculated pulse wave signal PPG, a blood pressure measurement value, and blood pressure information to the display controller 24. In some embodiments, as described with referenced to FIGS. 6 and 18, the processor 710 outputs to the display controller 24 a first user interface displaying an image of a generation section of the pulse wave signal PPG and an image of a regeneration section P3 of the pulse wave signal PPG, and a second user interface displaying an image of a pressure measurement value and an image of a pressure request range U24. Accordingly, in some embodiments, the display controller 24 outputs image information of at least one of the first user interface and the second user interface to the display panel 10.

According to some aspects, the display controller 24 receives the image signal supplied from the processor 710. According to some aspects, the display controller 24 generates a scan control signal GCS for controlling an operation timing of the scan driver 21, an emission control signal for controlling an operation timing of the emission driver 23, and a data control signal DCS for controlling an operation timing of a data driver 22. According to some aspects, the display controller 24 outputs image data DATA and a data control signal DCS to the data driver 22. According to some aspects, the display controller 24 outputs the scan control signal GCS to the scan driver 21 and outputs the emission control signal to the emission driver 23.

According to some aspects, the display controller 24 is electrically connected to the display panel 10 and/or the processor 710 through electrical lines. According to some aspects, the display controller 24 is connected to the display panel 10 and/or the processor 710 through a communication network. In at least one embodiment, at least a portion of the display controller 24 is implemented as a driving chip that is directly attached to the display panel 10.

According to some aspects, the data driver 22 receives the image data DATA and the data control signal DCS from the display controller 24. According to some aspects, the data driver 22 converts the image data DATA into an analog data voltage according to the data control signal DCS. According to some aspects, the data driver 22 outputs the converted analog data voltage to data lines DL in synchronization with scan signals.

According to some aspects, the scan driver 21 generates scan signals according to the scan control signal GCS, respectively, and sequentially outputs the scan signals to scan lines SL1 to SLn.

According to some aspects, the display device 1 includes a driving voltage, a common voltage, and a source voltage line. According to some aspects, the source voltage line includes a driving voltage line and a common voltage line. According to some aspects, the driving voltage is be a high potential voltage for driving light-emitting elements and photoelectric conversion elements, and the common voltage is a low-potential voltage for driving the light-emitting elements and the photoelectric conversion elements. In some embodiments, the driving voltage accordingly has a higher potential than the common voltage.

According to some aspects, a display control signal includes the scan control signal GCS, the data control signal DCS, and the emission control signal ECS. According to some aspects, the display control signal is output from the scan driver 21 and the data driver 22.

According to some aspects, the emission driver 23 generates emission signals Ek_1 according to the emission control signal ECS and sequentially outputs the emission signals Ek_1 to emission lines ELL. Referring to FIG. 2, in at least one embodiment, the emission driver 23 is disposed externally to the scan driver 21. In some embodiments, the emission driver 23 is included in the scan driver 21.

According to some aspects, the data driver 22 and the display controller 24 are included in the panel driving circuit 20. In some embodiments, the panel driving circuit 20 controls an operation of the display panel 10. According to some aspects, the data driver 22 and the display controller 24 are formed separately or together as one or more integrated circuits (ICs) and are mounted on the panel driving circuit 20.

According to some aspects, each of the plurality of pixels PX described with reference to FIG. 1 are connected to at least one of the scan lines SL1 to SLn, to at least one of the data lines DL, and to at least one of the emission lines ELL. According to some aspects, each of the plurality of photo-sensors PS described with reference to FIG. 1 are connected to at least one of the scan lines SL1 to SLn and to at least one of lead-out lines ROL.

According to some aspects, the scan lines SL1 to SLn connect the scan driver 21 to the plurality of pixels PX and to the plurality of photo-sensors PS, respectively. According to some aspects, the scan lines SL1 to SLn provide the scan signals output from the scan driver 21 to the plurality of pixels PX, respectively.

According to some aspects, a plurality of data lines DL connect the data driver 22 to the plurality of pixels PX, respectively. According to some aspects, the plurality of data lines DL provide the image data output from the data driver 22 to the plurality of pixels PX, respectively.

According to some aspects, a plurality of emission lines ELL connect the emission driver 23 to the plurality of pixels PX, respectively. According to some aspects, the plurality of emission lines ELL provide the emission control signals output from the emission driver 23 to the plurality of pixels PX, respectively.

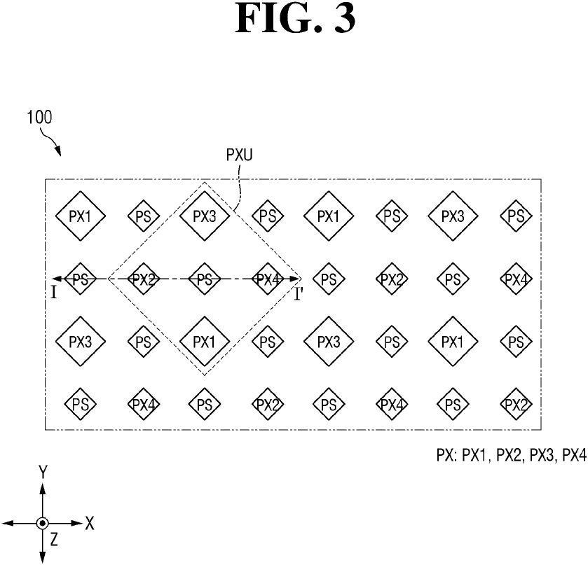

FIG. 3 is a plan layout view of pixels and photo-sensors of a display cell according to at least one embodiment.

Referring to FIG. 3, a plurality of pixels PX and a plurality of photo-sensors PS are disposed in a display cell 100. According to some aspects, the pixels PX and the photo-sensors PS are disposed in a repeating pattern.

According to some aspects, the plurality of pixels PX includes a first pixel PX1, a second pixel PX2, a third pixel PX3, and a fourth pixel PX4. In some embodiments, the first pixel PX1 emits light of a red wavelength, the second pixel PX2 and the fourth pixel PX4 emit light of a green wavelength, and the third pixel PX3 emits light of a blue wavelength. According to some aspects, the plurality of pixels PX include a plurality of emission areas emitting light, respectively. According to some aspects, the plurality of photo-sensors PS include a plurality of light-sensing areas sensing light incident thereon.

According to some aspects, the first pixel PX1, the second pixel PX2, the third pixel PX3, the fourth pixel PX4, and the plurality of photo-sensors PS are alternately arranged in the first direction X and in the second direction Y. For example, in at least one embodiment, first pixels PX1 and third pixels PX3 are alternately arranged in a first row along the first direction X, second pixels PX2 and fourth pixels PX4 are alternately arranged along the first direction in a second row adjacent to the first row, and photo-sensors PS are disposed in the first and second rows between proximate pairs of pixels PX and between pixels PX and an edge of the display cell 100. According to some aspects, pixels PX included in the first row are misaligned with pixels PX included in the second row in the first direction X. For example, in some embodiments, a photo-sensor PS included in the first row is disposed above a pixel PX included in the second row with respect to the second Y direction, and a photo-sensor PS included in the second row is disposed below a pixel PX included in the first row with respect to the second Y direction. According to some aspects, arrangements of the first row and the second row are repeated up to an n-th row.

According to some aspects, the photo-sensors PS are spaced apart from each other by the first pixels PX1 and the third pixels PX3 forming the first row. Accordingly, in some embodiments, first pixels PX1, photo-sensors PS, and third pixels PX3 may be alternately arranged along the first direction X. According to some aspects, the photo-sensors PS are spaced apart from each other by the second pixels PX2 and the fourth pixels PX4 forming the second row. Accordingly, in some embodiments, second pixels PX2, photo-sensors PS, and fourth pixels PX4 may be alternately arranged along the first direction X. According to some aspects, a number of photo-sensors PS included in the first row may be the same as a number of photo-sensors PS included in the second row. According to some aspects, arrangements of the first row and the second row are repeated up to the n-th row.

According to some aspects, the photo-sensors PS are respectively disposed between the second pixels PX2 and the fourth pixels PX4 forming the second row and are not respectively disposed between the first pixels PX1 and the third pixels PX3 forming the first row. For example, in some embodiments, the photo-sensors PS are omitted from the first row.

According to some aspects, sizes of emission areas of respective pixels PX are different from each other. In some embodiments, sizes of emission areas of the second pixel PX2 and the fourth pixel PX4 are smaller than sizes of emission areas of the first pixel PX1 or the third pixel PX3. Referring to FIG. 43, in at least one embodiment, the pixels PX have a rhombic shape. According to some aspects, the pixels PX have a rectangular shape, an octagonal shape, a circular shape, or another polygonal shape.

According to some aspects, a pixel unit PXU refers to a group of color pixels capable of expressing a gradation. According to some aspects, a pixel unit PXU includes a first pixel PX1, a second pixel PX2, a third pixel PX3, and a fourth pixel PX4.

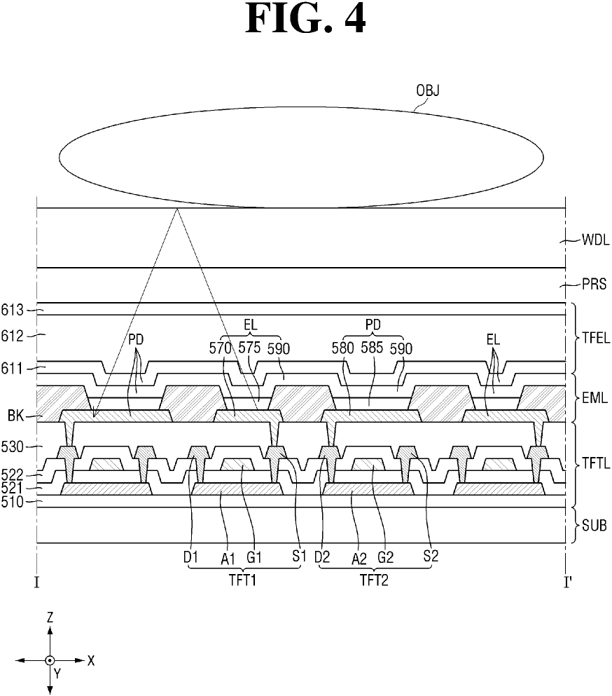

FIG. 4 is a cross-sectional view taken along line I-I′ of FIG. 3.

Referring to FIG. 4, a buffer layer 510 is disposed on a substrate SUB. According to some aspects, the buffer layer 510 includes silicon nitride, silicon oxide, silicon oxynitride, or the like. According to some aspects, a gate insulating layer 521 is disposed above the buffer layer 510. According to some aspects, an interlayer insulating film 522 is disposed above the gate insulating layer 521.

According to some aspects, a first thin film transistor TFT1 and a second thin film transistor TFT2 are disposed on the buffer layer 510. According to some aspects, the first and second thin film transistors TFT1 and TFT2 respectively include semiconductor layers A1 and A2, gate electrodes G1 and G2, source electrodes S1 and S2, and drain electrodes D1 and D2. In some embodiments, the gate insulating layer 521 is disposed on the semiconductor layers A1 and A2. In some embodiments, the gate electrodes G1 and G2 are disposed on the gate insulating layer 521. In some embodiments, the interlayer insulating film 522 at least partially covers each of the semiconductor layers A1 and A2 and each of the gate electrodes G1 and G2. In some embodiments, the drain electrodes D1 and D2 are disposed on the interlayer insulating film 522.

According to some aspects, the semiconductor layers A1 and A2 form channels of the first thin film transistor TFT1 and of the second thin film transistor TFT2, respectively. According to some aspects, the semiconductor layers A1 and A2 include polycrystalline silicon. According to some aspects, the semiconductor layers A1 and A2 include single crystal silicon, low-temperature polycrystalline silicon, amorphous silicon, or an oxide semiconductor. According to some aspects, the oxide semiconductor includes a binary compound (ABₓ), a ternary compound (ABₓC_{y}), or a quaternary compound (ABₓC_{y}D_{z}), each of the binary compound, the ternary compound, and the quaternary compound including, for example, indium, zinc, gallium, tin, titanium, aluminum, hafnium (Hf), zirconium (Zr), magnesium (Mg), or the like. According to some aspects, the semiconductor layers A1 and A2 respectively include a channel region, a source region, and a drain region. According to some aspects, one or more of the channel region, the source region, and the drain region is doped with impurities.

According to some aspects, the gate insulating layer 521 is disposed on the semiconductor layers A1 and A2. According to some aspects, the gate insulating layer 521 electrically insulates a first gate electrode G1 and a first semiconductor layer A1 from each other and electrically insulates a second gate electrode G2 and a second semiconductor layer A2 from each other. According to some aspects, the gate insulating layer 521 includes an insulating material such as silicon oxide (SiOₓ), silicon nitride (SiNₓ), or metal oxide.

According to some aspects, the first gate electrode G1 of the first thin film transistor TFT1 and the second gate electrode G2 of the second thin film transistor TFT2 are disposed on the gate insulating layer 521. According to some aspects, the gate electrodes G1 and G2 are formed above the channel regions of the semiconductor layers A1 and A2. In an example, in some embodiments, the gate electrodes G1 and G2 are formed on positions of the gate insulating layer 521 overlapping the channel regions.

According to some aspects, the interlayer insulating film 522 is disposed on the gate electrodes G1 and G2. According to some aspects, the interlayer insulating film 522 includes an inorganic insulating material such as silicon oxide (SiOₓ), silicon nitride (SiNₓ), silicon oxynitride, hafnium oxide, or aluminum oxide. According to some aspects, the interlayer insulating film 522 includes a plurality of insulating films and a conductive layer disposed between proximate pairs of the insulating films and forming a capacitor second electrode.

According to some aspects, the source electrodes S1 and S2 and the drain electrodes D1 and D2 are disposed on the interlayer insulating film 522. According to some aspects, a first source electrode S1 of the first thin film transistor TFT1 is electrically connected to the drain region of the first semiconductor layer A1 through a contact hole penetrating through the interlayer insulating film 522 and the gate insulating layer 521. According to some aspects, a second source electrode S2 of the second thin film transistor TFT2 is electrically connected to the drain region of the second semiconductor layer A2 through a contact hole penetrating through the interlayer insulating film 522 and the gate insulating layer 521.

According to some aspects, each of the source electrodes S1 and S2 and the drain electrodes D1 and D2 includes one or more metals selected from a group comprising aluminum (Al), molybdenum (Mo), platinum (Pt), palladium (Pd), silver (Ag), magnesium (Mg), gold (Au), nickel (Ni), neodymium (Nd), iridium (Ir), chromium (Cr), calcium (Ca), titanium (Ti), tantalum (Ta), tungsten (W), and copper (Cu).

According to some aspects, a planarization layer 530 is formed on the interlayer insulating film 522 and at least partially covers each of the source electrodes S1 and S2 and the drain electrodes D1 and D2. According to some aspects, the planarization layer 530 includes an organic insulating material or the like. According to some aspects, the planarization layer 530 has a flat surface and includes contact holes exposing any one of the source electrodes S1 and S2 and any one of the drain electrodes D1 and D2.

According to some aspects, a light-emitting element layer EML is disposed on the planarization layer 530. According to some aspects, the light-emitting element layer EML includes a light-emitting element EL, a photoelectric conversion element PD, and a bank layer BK. According to some aspects, the light-emitting element EL includes a pixel electrode 570, an emission layer 575, and a common electrode 590, and the photoelectric conversion element PD includes a first electrode 580, a photoelectric conversion layer 585, and a common electrode 590.

According to some aspects, the pixel electrode 570 of the light-emitting element EL is disposed on the planarization layer 530. According to some aspects, the pixel electrode 570 is provided for each pixel PX. According to some aspects, the pixel electrode 570 is connected to the first source electrode S1 and/or the first drain electrode D1 of the first thin film transistor TFT1 through a contact hole penetrating through the planarization layer 530.

According to some aspects, the pixel electrode 570 of the light-emitting element EL includes a single-layer structure including molybdenum (Mo), titanium (Ti), copper (Cu), or aluminum (Al), or includes a stacked film structure including, for example, a multilayer structure of ITO/Mg, ITO/MgF, ITO/Ag, or ITO/Ag/ITO including indium-tin-oxide (ITO), indium-zinc-oxide (IZO), zinc oxide (ZnO), or indium oxide (In₂O₃), and silver (Ag), magnesium (Mg), aluminum (Al), platinum (Pt), lead (Pb), gold (Au), or nickel (Ni).

According to some aspects, the first electrode 580 of the photoelectric conversion element PD is disposed on the planarization layer 530. According to some aspects, the first electrode 580 is provided for each photo-sensor PS. According to some aspects, the first electrode 580 is connected to the second source electrode S2 or the second drain electrode D2 of the second thin film transistor TFT2 through a contact hole penetrating through the planarization layer 530.

According to some aspects, the first electrode 580 of the photoelectric conversion element PD includes a single-layer structure including molybdenum (Mo), titanium (Ti), copper (Cu), or aluminum (Al), or includes a multilayer structure including, for example, ITO/Mg, ITO/MgF, ITO/Ag, or ITO/Ag/ITO.

According to some aspects, the bank layer BK is disposed on the pixel electrode 570 and the first electrode 580. According to some aspects, the bank layer BK includes openings formed in areas overlapping the pixel electrodes 570 and exposing the pixel electrodes 570. In some embodiments, an area in which an exposed pixel electrode 570 and the emission layer 575 overlap each other is an emission area emitting different light according to the respective pixel PX.

According to some aspects, the bank layer BK includes openings formed in areas at least partially overlapping the first electrodes 580 and at least partially exposing the first electrodes 580. According to some aspects, the openings exposing the first electrodes 580 provide spaces in which the photoelectric conversion layers 585 of the respective photo-sensors PS are formed. In some embodiments, areas in which the exposed first electrodes 580 and the photoelectric conversion layers 585 overlap each other are light-sensing parts RA.

According to some aspects, the bank layer BK includes an organic insulating material such as a polyacrylates resin, an epoxy resin, a phenolic resin, a polyamides resin, a polyimides resin, an unsaturated polyesters resin, a polyphenyleneethers resin, a polyphenylenesulfides resin, or benzocyclobutene (BCB). According to some aspects, the bank layer BK includes an inorganic material such as silicon nitride.

According to some aspects, the emission layers 575 are disposed on the pixel electrodes 570 of the light emitting elements EL exposed by the openings of the bank layer BK. According to some aspects, an emission layer 575 includes a high molecular material or a low molecular material, and respectively emits red, green, or blue light according to a corresponding pixel PX. According to some aspects, the light emitted from the emission layer 575 contributes to a display of an image or functions as a light source incident on the photo-sensor PS. For example, in some embodiments, a light of a green wavelength emitted from an emission layer 575 of a second pixel PX2 or a fourth pixel PX4 may be incident on the light sensing areas of the photo-sensors PS.

According to some aspects, the emission layer 575 is formed of an organic material, a hole injecting layer (HIL) and a hole transporting layer (HTL) is disposed at a lower portion of each emission layer 575, and an electron injecting layer (EIL) and an electron transporting layer (ETL) are stacked at an upper portion of each emission layer 575. Each of these layers may be a single layer or a multiple layer made of an organic material.

According to some aspects, the photoelectric conversion layers 585 are disposed on the first electrodes 580 of the photoelectric conversion elements PD exposed by the openings of the bank layer BK. According to some aspects, areas in which the exposed first electrodes 580 and the photoelectric conversion layers 585 overlap each other are light-sensing areas of the respective photo-sensors PS. According to some aspects, a photoelectric conversion layer 585 generates photocharges in proportion to light incident on the photoelectric conversion layer 585. According to some aspects, the incident light is light that is emitted from an emission layer 575 and then reflected to enter the photoelectric conversion layer 585. According to some aspects, the incident light is light that is provided from any light source. According to some aspects, charges generated and accumulated in the photoelectric conversion layer 585 are converted into electrical signals used for sensing.

According to some aspects, the photoelectric conversion layer 585 includes an electron donating material and an electron accepting material. According to some aspects, the electron donating material generates donor ions in response to light and the electron accepting material generates acceptor ions in response to light. According to some aspects, the photoelectric conversion layer 585 is formed of an organic material, the electron donating material includes a compound such as subphthalocyanine (SubPc), dibutylphosphate (DBP), or the like. According to some aspects, the electron accepting material includes a compound such as fullerene, a fullerene derivative, perylene diimide, or the like.

According to some aspects, the photoelectric conversion layer 585 is formed of an inorganic material, and the photoelectric conversion element PD is a PN-type or a PIN-type phototransistor. For example, the photoelectric conversion layer 585 may include an N-type semiconductor layer, an I-type semiconductor layer stacked on the N-type semiconductor layer, and a P-type semiconductor layer stacked on the I-type semiconductor layer.

According to some aspects, the photoelectric conversion layer 585 is formed of an organic material, a hole injecting layer (HIL) and a hole transporting layer (HTL) is disposed at a lower portion of a photoelectric conversion layer 585, and an electron injecting layer (EIL) and an electron transporting layer (ETL) are stacked at an upper portion of the photoelectric conversion layer 585. According to some aspects, each of the HIl, the HTL, the EIL, and the ETL includes a single layer or multiple layers including an organic material.

According to some aspects, the common electrode 590 is disposed on the emission layers 575, the photoelectric conversion layers 585, and the bank layer BK. According to some aspects, the common electrode 590 is disposed throughout the plurality of pixels PX and the plurality of photo-sensors PS and at least partially covers the emission layers 575, the photoelectric conversion layers 585, and the bank layer BK. According to some aspects, the common electrode 590 includes a material layer having a small work function. For example, in some embodiments, the material layer includes Li, Ca, LiF/Ca, LiF/Al, Al, Mg, Ag, Pt, Pd, Ni, Au, Nd, Ir, Cr, BaF, Ba, or compounds or mixtures thereof (e.g., a mixture of Ag and Mg, etc.). According to some aspects, the common electrode 590 includes transparent metal oxide, such as indium-tin-oxide (ITO), indium-zinc-oxide (IZO), or zinc oxide (ZnO).

According to some aspects, the common electrode 590 is commonly disposed on an emission layer 575 and a photoelectric conversion layer 585. In this case, a cathode electrode of a corresponding light-emitting element EL and a sensing cathode electrode of a corresponding photoelectric conversion element PD are electrically connected to each other. For example, in some embodiments, a common voltage line connected to the cathode electrode of the light emitting element EL is also connected to the sensing cathode electrode of the photoelectric conversion element PD.

According to some aspects, an encapsulation layer TFEL is disposed on the light emitting element layer EML. According to some aspects, the encapsulation layer TFEL includes at least one inorganic film that reduces a penetration of oxygen or moisture into each of the emission layers 575 and the photoelectric conversion layers 585. According to some aspects, the encapsulation layer TFEL includes at least one organic film that protects each of the emission layer 575 and the photoelectric conversion layer 585 from foreign materials such as dust. For example, in some embodiments, the encapsulation layer TFEL includes a first inorganic film 611, an organic film 612 stacked on the first inorganic film 611, and a second inorganic film 613 stacked on the organic film 612. According to some aspects, each of the first inorganic film 611 and the second inorganic film 613 are formed as a multiple film in which one or more inorganic films of a silicon nitride layer, a silicon oxynitride layer, a silicon oxide layer, a titanium oxide layer, and an aluminum oxide layer are alternately stacked. According to some aspects, the organic film 612 includes an acrylic resin, an epoxy resin, a phenolic resin, a polyamide resin, a polyimide resin, or the like.

According to some aspects, a pressure sensing layer PRS is disposed on the encapsulation layer TFEL. According to some aspects, the pressure sensing layer PRS is a panel or a film, and is attached to the encapsulation layer TFEL via a bonding layer such as a pressure-sensitive adhesive (PSA). According to some aspects, the pressure-sensing layer PRS is positioned on a light-emission path of the light-emitting element layer EML. In some embodiments, the pressure-sensing layer PRS is therefore transparent.

According to some aspects, the pressure-sensing layer PRS senses a pressure applied to the display device 1. For example, in some embodiments, when a user or the like provides a touch input to an upper surface of the display device 1, the pressure-sensing layer PRS senses a pressure caused by the touch input. According to some aspects, a pressure-sensing electrode of the pressure-sensing layer PRS is directly formed on a touch layer. In this case, the pressure-sensing layer PRS is included in the display panel 10 together with the display layer 120 and the touch layer.

According to some aspects, a window WDL is disposed on the pressure-sensing layer PRS. According to some aspects, the window WDL is disposed on the display device 1 and protects components of the display device 1 during and/or after a cutting process and a module process of the display cell 100 are performed. In some embodiments, the window WDL includes a transparent or a semi-transparent material, such as glass or plastic.

Referring to FIG. 4, a portion of a user OBJ touches the window WDL of the display device 1. In some embodiments, the portion of the user OBJ is a finger OBJ. When the finger OBJ touches an upper surface of the window WDL, light output from the emission areas of the pixels PX may be reflected from the finger OBJ. In this case, a rate of blood flow in the finger OBJ may change according to a pressure in a blood vessel of the finger OBJ. As the rate of blood flow in the finger OBJ may be determined based on a difference in an amount of light reflected from the finger OBJ to a photo-sensor PS, and the rate of blood flow in the finger OBJ corresponds to a blood pressure of the user, the blood pressure of the user is therefore able to be measured by the display device 1 using the photo-sensor PS and the pressure sensing layer PRS.

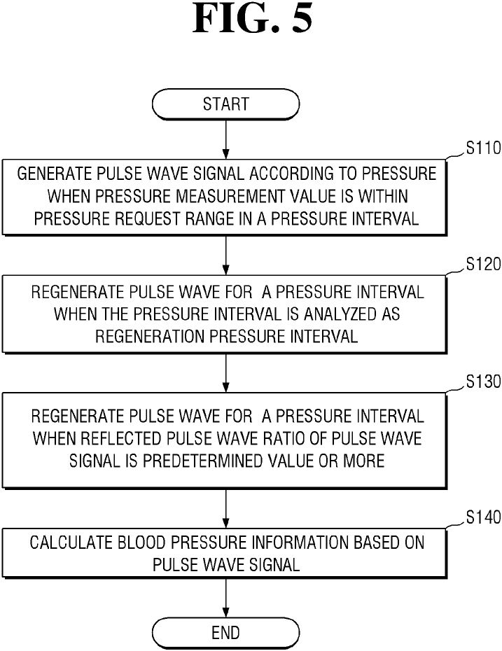

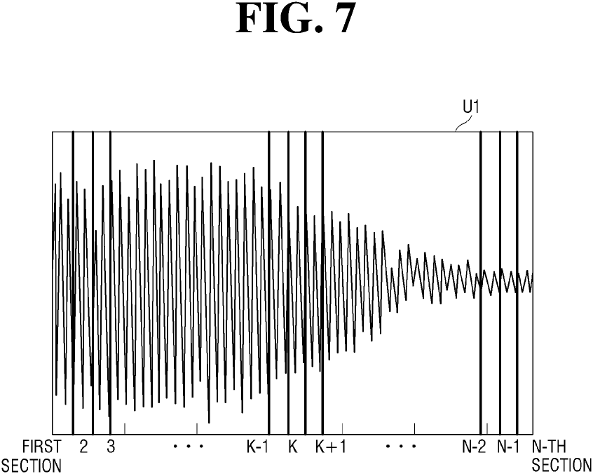

FIG. 5 is a flowchart illustrating process for blood pressure measurement according to at least one embodiment. FIG. 6 is a plan view illustrating a user interface of the display device of FIG. 1 according to at least one embodiment. FIG. 7 is an enlarged plan view of a first user interface of FIG. 6.

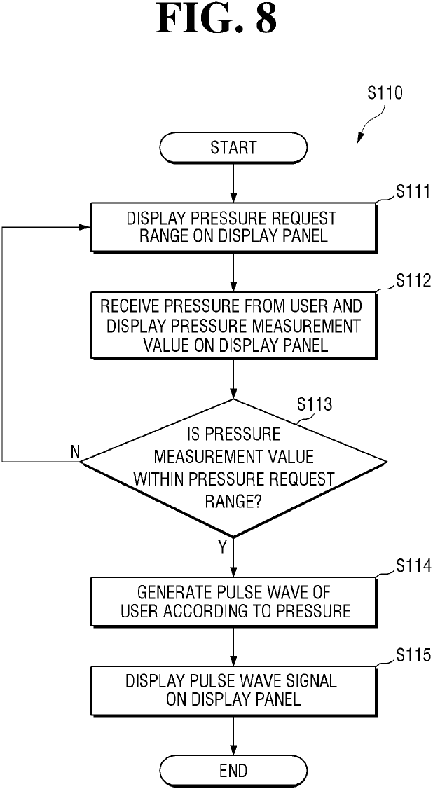

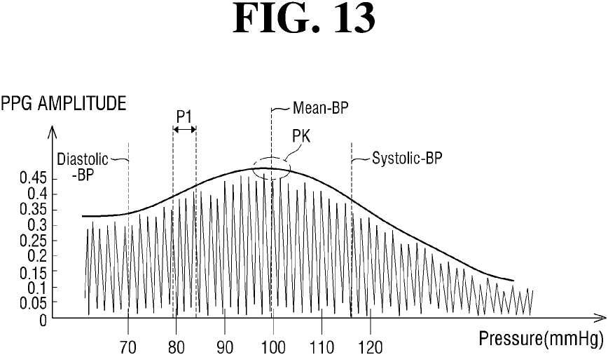

Referring to FIG. 5, in operation S110, when a pressure measurement value (e.g., a value of a pressure applied by a user input to the display device 1) is within a preset pressure request range U24 described with reference to FIG. 6 corresponding to a pressure interval of the first to N-th pressure intervals, a processor 710 described with reference to FIG. 2 generates a pulse wave signal PPG as described with reference to FIGS. 13-14 and 16-17 based on an amount of light sensed by the photo-sensor PS and an optical signal corresponding to the amount of light. Examples or aspects of operation S110 are described in further detail with reference to FIG. 8.

Referring to FIGS. 5 to 7, according to some aspects, the display panel 10 includes a first area and a second area. According to some aspects, the first area includes a first user interface U1. According to some aspects, the first area of the display panel 10 displays values of the pulse wave signal PPG via a first portion of the first user interface U1. For example, in some embodiments, the values of the pulse wave signal PPG are generated by the processor 710, and the first portion of the first user interface U1 displays the values of the pulse wave signal PPG after they are generated.

In addition, in some embodiments, the first area of the display panel 10 displays images of values of the pulse wave signal PPG that are being generated via a second portion of the first user interface U1. For example, in some embodiments, as the values of the pulse wave signal PPG are generated by the processor 710, the second portion of the first user interface U1 displays images of the values of the pulse wave signal PPG as they is generated. In addition, in some embodiments, the first area of the display panel 10 displays a third portion of the first user interface U1. In some embodiments, the third portion of the first user interface U1 is reserved for displaying images of values of the pulse wave signal PPG that are scheduled to be generated. Accordingly, a user may respectively confirm that the pulse wave signal PPG has been generated, is being generated, and is scheduled to be generated in real time by viewing the first through third portions of the first user interface U1.

According to some aspects, the processor 710 determines the first to N-th pressure intervals for generating the pulse wave signal PPG. According to some aspects, each of the first to N-th pressure intervals is an interval of pressure values that correspond to one or more values of pressure applied to the display device 1 by a user input. In some embodiments, the pressure applied to the display device 1 is measured in terms of millimeters of mercury (mmHg).. According to some embodiments, an interval of pressure values is predetermined. In an example, in some embodiments, any one of the first to N-th pressure intervals is a K-th pressure interval, and predetermined bounding values of the K-th pressure interval may be for example, about 2 mmHg and about 5 mmHg, or the K-th pressure interval may include different bounding values.

According to some aspects, values of the pulse wave signal PPG are sequentially generated according to the first to N-th pressure intervals. For example, in some embodiments, any one of the first to N-th pressure intervals is referred to as a K-th pressure interval, a previous pressure interval adjacent to the K-th pressure interval is referred to as a K-1-th pressure interval, and a next pressure interval adjacent to the K-th pressure interval is referred to as a K+1-th pressure interval. Accordingly, in some embodiments, the processor 710 generates K-1-th values of the pulse wave signal PPG corresponding to the K-1-th pressure interval, then generates K-th values of the pulse wave signal PPG corresponding to the K-th pressure interval, and then generates K+1-th values of the pulse wave signal PPG corresponding to the K+1-th pressure interval. For example, in some embodiments, values of the pulse wave signal PPG are sequentially generated according to the K-1-th pressure interval, the K-th pressure interval, and the K+1-th pressure interval, such that, for example, when the display device 1 receives a touch input that provides a pressure measurement value within the K-1-th pressure interval, the processor 710 generates a value of the pulse wave signal PPG corresponding to the pressure measurement value.

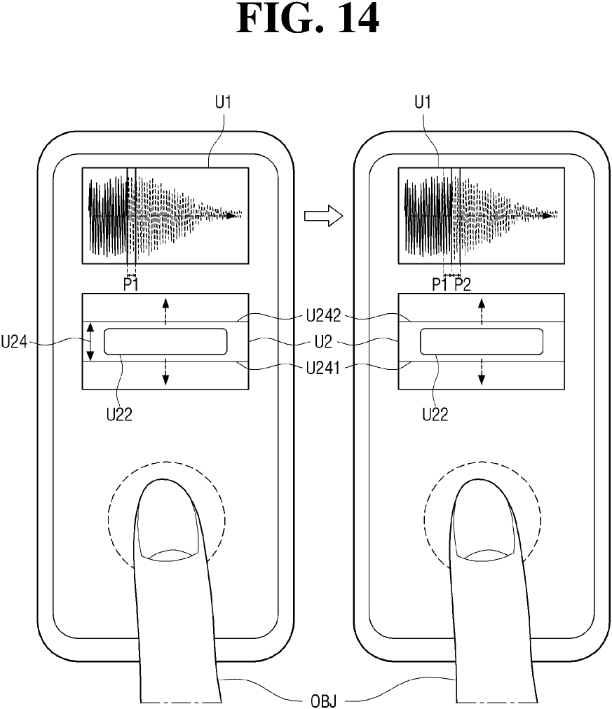

Accordingly, in some embodiments, the first user interface U1 includes first to N-th portions that respectively display images of values of the pulse wave signal PPG corresponding to the first to N-th pressure intervals. The first user interface U1 is described in further detail with reference to FIG. 14.

According to some aspects, the second area of the display panel 10 displays the second user interface U2. According to some aspects, the second area of the display panel 10 displays a pressure request range U24 for pulse wave measurement and a pressure measurement value sensed by the pressure sensor via the second user interface U2. According to some aspects, the second area of the display panel 10 displays pressure request ranges U24 and pressure measurement values via first to N-th portions of the second user interface U2 corresponding to the first to N-th pressure intervals. Accordingly, in some embodiments, the second user interface U2 displays pressure information corresponding to each of the first to N-th pressure intervals, allowing a user to confirm that they are providing suitable pressure for pulse wave measurement within each pressure interval in real time.

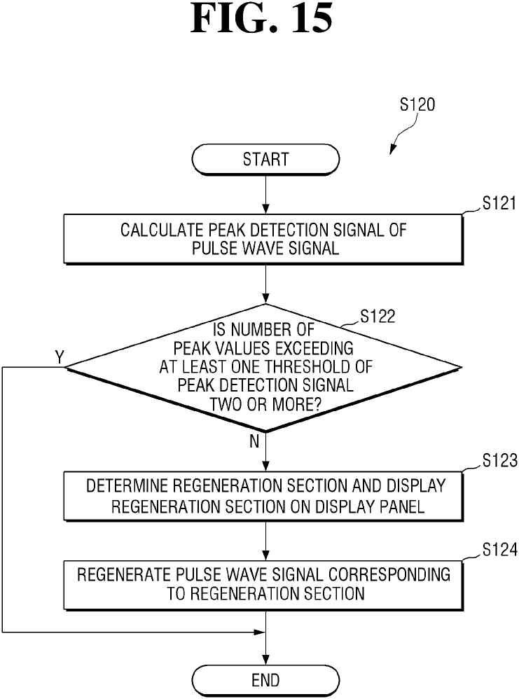

Referring to FIG. 5, in operation S120, a pressure interval of the first to N-th pressure intervals is analyzed as a regeneration pressure interval P3 as described with reference to FIGS. 15-19 and the processor 710 regenerates the pulse wave signal PPG for the pressure interval according to an amount of light sensed by the photo-sensor PS corresponding to the at least one section and according to an optical signal corresponding to the amount of light. Examples or aspects of operation S120 are described in further detail with reference to FIG. 15.

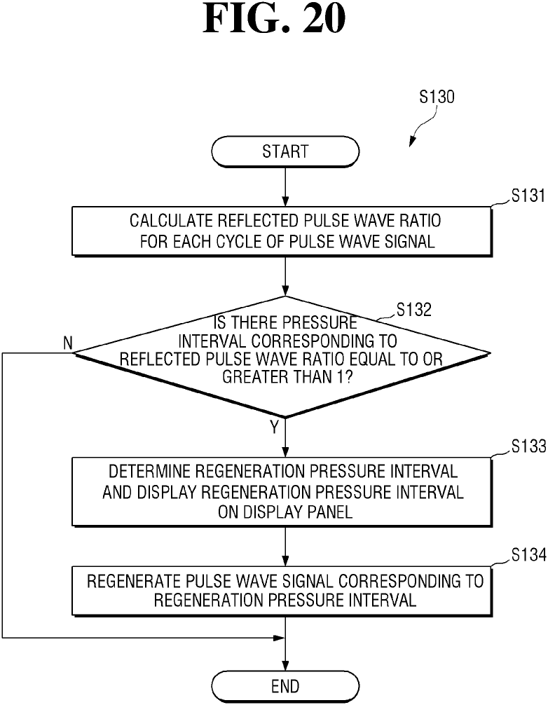

In operation S130, when a reflected pulse wave ratio RI as described with reference to FIGS. 20 to 23 corresponding to a pressure interval of the first to N-th pressure intervals is a predetermined value or more, the processor 710 regenerates the pulse wave signal PPG for the pressure interval. Examples of or aspects of operation S130 are described in further detail with reference to FIG. 20.

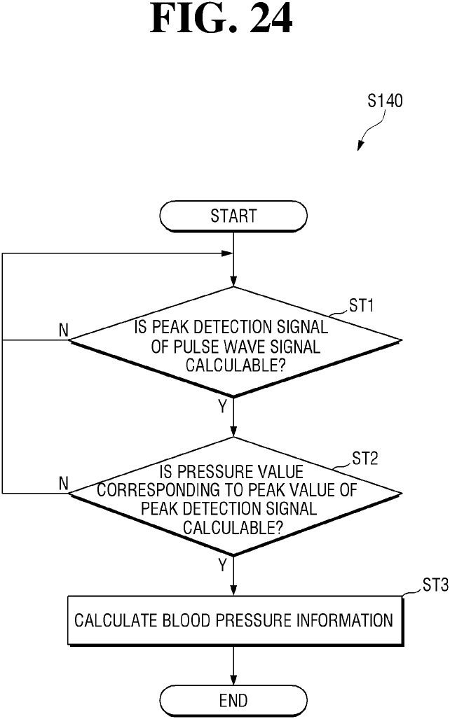

In operation S140, the processor 710 calculates blood pressure information based on the pulse wave signal PPG as described with reference to FIG. 24.

According to at least one embodiment, the pulse wave signal PPG is regenerated for pressure interval of the first to N-th pressure intervals. In addition, in some embodiments, the display device 1 accurately measures blood pressure information based on the generated and regenerated pulse wave signals PPG.

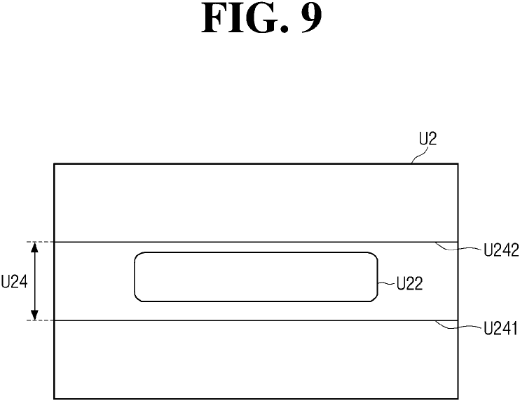

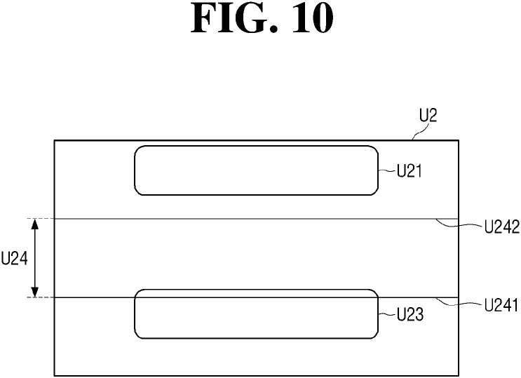

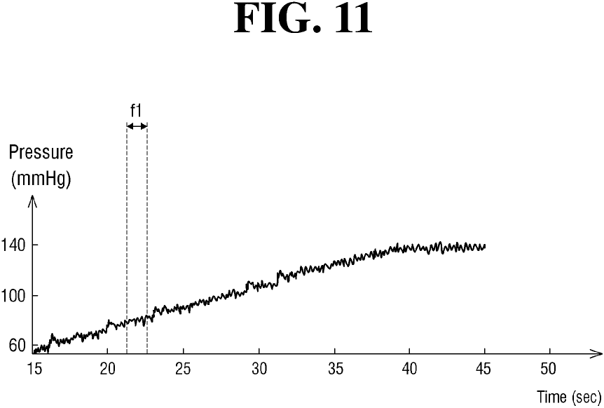

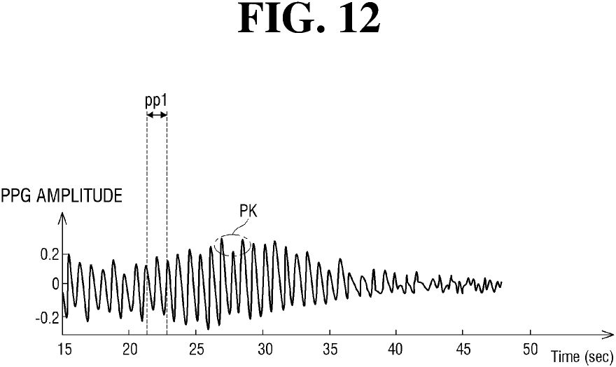

A process for generating a pulse wave signal PPG and displaying a user interface by the display panel 10 according to at least one embodiment is described in detail with reference to FIGS. 8 to 14. FIG. 8 is a flowchart illustrating a process for generating a pulse wave signal according to at least one embodiment. FIGS. 9 and 10 are enlarged plan views of a second user interface of the display device of FIG. 1 according to at least one embodiment. FIG. 11 is a graph illustrating a pressure measurement value according to a pressure applying time. FIG. 12 is a graph illustrating a pulse wave signal according to a pressure applying time. FIG. 13 is a graph illustrating a relationship between a pressure and a pulse wave signal. FIG. 14 is a plan view illustrating a user interface of the display device of FIG. 1 according to an embodiment.

Referring to FIG. 8, in operation S111, the processor 710 displays the pressure request range U24 on the display panel 10. Referring to FIGS. 8 and 9, according to some aspects, a pressure request range U24 is predetermined for each of the first to N-th pressure intervals and is displayed on the display panel 10. For example, the respective pressure request range U24 may gradually increase for the first to N-th pressure intervals. For example, when a pulse wave is measured in the K-th pressure interval (e.g., any one of the first to N-th pressure intervals), the pressure request range U24 corresponding to the K+1-th pressure interval may be greater than the pressure request range U24 corresponding to the K-th pressure interval.

According to some aspects, the pressure request range U24 for each pressure interval includes a first requested pressure U241 and a second requested pressure U242 higher than the first requested pressure U241 and refers to a difference in pressure between the first requested pressure U241 and the second requested pressure U242. For example, in some embodiments, for each of the first to N-th pressure intervals, a second requested pressure U242 may be greater than a first requested pressure U241 by about 2 mmHg to about 5 mmHg. In an example, in some embodiments, in the K-th pressure interval (e.g., any one of the first to N-th pressure intervals), a second requested pressure U242 may be about 85 mmHg and a first requested pressure U241 may be about 80 mmHg. Accordingly, this case, in the K-th pressure interval, the pressure request range U24 may be about 80 mmHg to about 85 mmHg. According to some aspects, the pressure request range U24 for the K-th pressure interval may have a greater or a smaller value than about 80 mmHg.

According to some aspects, the pressure request range U24 is displayed via the second user interface U2 of the display panel 10. For example, in some embodiments, as illustrated in FIG. 9, each of the first requested pressure U241 and the second requested pressure U242 are displayed via the second user interface U2, and the pressure request range U24 that represents an interval between the first requested pressure U241 and the second requested pressure U242 is also displayed via the second user interface U2.

Referring to FIG. 8, in operation S112, a pressure sensor receives a pressure from a user via a touch input, and the pressure sensor measures a pressure measurement value for the pressure received from the user. In some embodiments, the display device 1 displays the pressure measurement value on the display panel 10.

Referring to FIGS. 8 to 10, the user may provide a touch input that applies a pressure corresponding to a pressure interval of the first to N-th pressure intervals to a position where the pressure sensor is disposed, and the pressure sensor measures the pressure measurement value for the pressure applied by the touch input of the user. In some embodiments, the pressure measurement value is measured in mmHg.

According to some aspects, the pressure measurement value is displayed by the display device 1 via the second user interface U2. in an example, as illustrated by FIG. 9, when the pressure sensor measures a second pressure measurement value U22 corresponding to a value in the K-th pressure interval, the second pressure measurement value U22 may be displayed via the second user interface U2. In an example, as illustrated by FIG. 10, when the pressure sensor measures a first pressure measurement value U21 greater than the second pressure measurement value U22 corresponding to the K-th pressure interval, the first pressure measurement value U21 may be displayed via the second user interface U2. In an example, when the pressure sensor measures a third pressure measurement value U23 smaller than the second pressure measurement value U22 corresponding to the K-th pressure interval, the third pressure measurement value U23 may be displayed in the second user interface U2.

Referring to FIG. 8, in operation S113, the processor 710 determines whether the pressure measurement value is within the preset pressure request range U24.

In some embodiments, as illustrated by FIG. 9, when the pressure sensor measures the second pressure measurement value U22 corresponding to the K-th pressure interval, the processor 710 determines that the second pressure measurement value U22 is within the pressure request range U24. For example, the processor 710 determines that the second pressure measurement value U22 has a value greater than the first requested pressure U241 and smaller than the second requested pressure U242.

In some embodiments, as illustrated by FIG. 10, the pressure sensor measures the first pressure measurement value U21 in the K-th pressure interval and the processor 710 determines that the first pressure measurement value U21 has a value that is not included in the pressure request range U24. For example, the processor 710 may determine that the first pressure measurement value U21 has a value greater than the first requested pressure U241 and greater than the second requested pressure U242.

In some embodiments, the pressure sensor measures the third pressure measurement value U23 corresponding to the K-th pressure interval and the processor 710 determines that the third pressure measurement value U23 has a value that is not included in the pressure request range U24. For example, the processor 710 may determine that the third pressure measurement value U23 has a value smaller than the first requested pressure U241 and smaller than the second requested pressure U242.

In operation S114, when the pressure measurement value is within the pressure request range U24 (S113: Y), the processor 710 generates the pulse wave signal PPG.

As described above, in some embodiments, when the pressure measurement value is within the pressure request range U24 corresponding to the K-th pressure interval, the processor 710 generates the pulse wave signal PPG corresponding to the K-th pressure interval.

A process for generating the pulse wave signal PPG is described in further detail with reference to FIGS. 11 to 13. According to some aspects, the processor 710 generates pressure data based on the pressure measurement value. For example, in some embodiments, when the user applies a pressure to a pressure sensor by providing a touch input to the display device 1, pressure measurement values measured by the pressure sensor may gradually increase, such that the pressure management values respectively correspond to the first to N-th pressure intervals, until a maximum pressure measurement value is measured. When the pressure measurement values (e.g., measurements of the amounts of pressure applied by a user’s touch input) increase, a blood vessel of the user may be constricted, such that a blood flow rate may be decreased or become zero. According to some aspects, the pressure data includes one or more pressure measurement values.

Referring to FIG. 11, in some embodiments, a pressure request range U24 corresponding to the K-th pressure interval may be about 80 mmHg to about 85 mmHg. For example, a K-th pressure measurement value f1 corresponding to the pressure request range U24 corresponding to the K-th pressure interval may be about 80 mmHg to about 85 mmHg.

According to some aspects, the pulse wave signal PPG is generated based on pulse wave information corresponding to a time. During a systole of a heart of a user, blood ejected from the left ventricle of the heart moves to peripheral tissues of the user, such that a blood volume in the arterial side of the heart increases. In addition, during the systole of the heart, red blood cells carry more oxyhemoglobin to the peripheral tissues of the user. During a diastole of the heart, there is partial suction of blood from the peripheral tissues towards the heart. In this case, according to some aspects, when a peripheral blood vessel of the user is irradiated with light emitted from the display pixel, the irradiated light may be absorbed by the peripheral tissue.

According to some aspects, absorbance of the light corresponds to a hematocrit and a blood volume of the user. In some embodiments, the absorbance has a maximum value during the systole of the heart and a minimum value during the diastole of the heart. According to some aspects, since the absorption is in inverse proportion to an amount of light incident on the photo-sensor PS, absorption at a corresponding point in time is estimated based on light-reception data of the amount of light incident on the photo-sensor PS, and accordingly, as illustrated in FIG. 12, pulse wave information according to a time is generated. For example, in some embodiments, the processor 710 generates K-th pulse wave information pp1 corresponding to the K-th pressure interval.

According to some aspects, the pulse wave information reflects a maximum value of the absorption during the systole of the heart, and reflects the minimum value of the absorption during the diastole of the heart. In some embodiments, the pulse wave vibrates according to a heartbeat cycle. Accordingly, in some embodiments, the pulse wave information reflects a change in a blood pressure of the user, as determined based on the heartbeat cycle.

Accordingly, referring to FIG. 13, in some embodiments, the processor 710 generates a pulse wave signal PPG based on the pressure data and the pulse wave information. For example, in some embodiments, when the display device receives a pressure from a user corresponding to the K-th pressure interval, the processor 710 generates a K-th pulse wave signal P1 based on the K-th pressure measurement value f1 and the K-th pulse wave information pp1.

According to some aspects, the processor 710 generates a pulse wave signal PPG corresponding to each of the first to N-th pressure intervals in a similar manner as generating the K-th pulse wave signal P1 corresponding to the K-th pressure interval.

Referring to FIG. 8, in operation S115, the pulse wave signal PPG is displayed on the display panel 10.

Referring to FIG. 14, according to some aspects, the first area of the display panel 10 displays images of the pulse wave signal PPG generated in the first to N-th pressure intervals via the first user interface U1. In some embodiments, the first user interface U1 displays images of a pulse wave signal PPG that is generated based on a pressure interval. For example, in some embodiments, the first user interface U1 displays images of values of the pulse wave signal PPG that have already been generated with respect to a previous pressure interval using a solid line and displays images of values of the pulse wave signal PPG that are being generated with respect to a current pressure interval using a dotted line so that the user may identify the whether the values of the pulse wave signal PPG are already generated or are being generated. In some embodiments, the first user interface U1 displays the images of values of the pulse wave signal PPG that have been generated with respect to a previous pressure interval using a dark color and displays images of the values of the pulse wave signal PPG that are being generated with respect to a current pressure interval using a light color so that the user may identify whether the values of the pulse wave signal PPG are already generated or are currently being generated.

According to some aspects, when the processor 710 generates a value of the pulse wave signal PPG corresponding to any one of the first to N-th pressure intervals, the first user interface U1 displays the pressure interval corresponding to the value of the pulse wave signal PPG. For example, in some embodiments, when the processor 710 generates a value of the K-th pulse wave signal PPG corresponding to the K-th pressure interval, the first user interface U1 changes a color of a portion of the image of the pulse wave signal PPG corresponding to the K-th pressure interval or performs shading on the portion of the image of the pulse wave signal PPG corresponding to the K-th pressure interval so that the K-th pressure interval may be identified.

Accordingly, in some embodiments, when the processor 710 continuously generates values of the pulse wave signal PPG corresponding to the first to N-th pressure intervals, the first user interface U1 continuously displays images of the pressure intervals corresponding to the generated values of the pulse wave signal PPG. For example, in some embodiments, when the processor 710 continuously generates values of the pulse wave signal PPG corresponding to the K-th pressure interval and the K+1-th pressure interval, the first user interface U1 continuously changes colors of portions of the images of the pulse wave signal PPG corresponding to the K-th pressure interval and the K+1-th pressure interval or performs shading on portions of the pulse wave signal PPG corresponding to the K-th pressure interval and the K+1-th pressure interval.

According to some aspects, the first user interface U1 may make any visual change to the images of the pulse wave signal PPG such that any values of the pulse wave signal PPG respectively corresponding to the first to N-th pressure intervals can be distinguished from each other.

When the pressure measurement value is not within the preset pressure request range U24 (S113: N), the user may re-apply a pressure to the pressure sensor so that the pressure measurement value exists within the pressure request range U24.

Accordingly, in some embodiments, when the processor 710 generates values of the pulse wave signal PPG corresponding to the first to N-th pressure intervals, the generated pulse wave signal PPG, the pressure request ranges U24, and the pressure measurement values are displayed on the display panel 10. Accordingly, in some embodiments, the user may identify the pulse wave signal PPG, the pressure request range U24, and the pressure measurement value in real time.

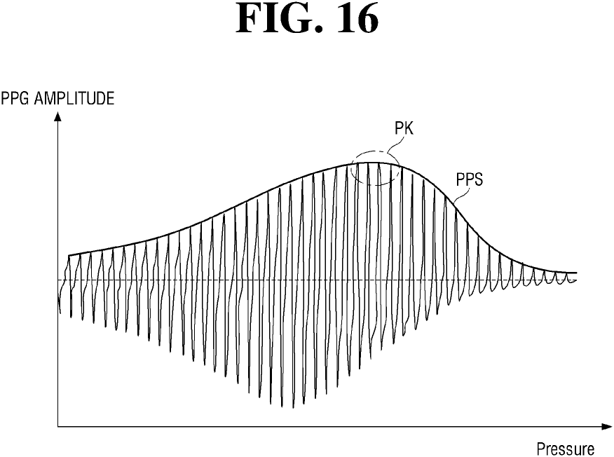

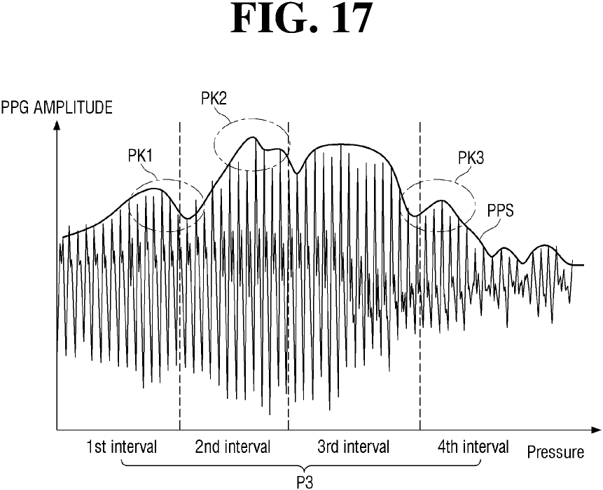

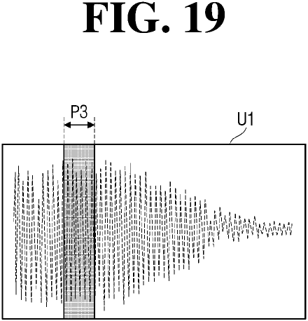

A process for regenerating the pulse wave signal PPG by the processor 710 is described in further detail with reference to FIGS. 15 to 19. FIG. 15 is a flowchart illustrating a process for regenerating a pulse wave signal according to at least one embodiment. FIGS. 16 and 17 are graphs illustrating pulse wave signals according to at least one embodiment. FIG. 18 is a plan view illustrating a user interface of the display device of FIG. 1 according to at least one embodiment. FIG. 19 is an enlarged plan view of a first user interface of FIG. 18.

Referring to FIG. 15, in operation S121, a peak detection signal PPS of the pulse wave signal PPG is calculated.

Referring to FIGS. 15 and 16, according to some aspects, the processor 710 generates the peak detection signal PPS using peak values of the pulse wave signal PPG. According to some aspects, the peak detection signal PPS is a signal corresponding to each peak value of one cycle of the pulse wave signal PPG. For example, in some embodiments, values of the pulse wave signal PPG generated based on each of the first to N-th pressure intervals include one or more peak values. According to some aspects, the processor 710 calculates the peak detection signal PPS to include the peak values of the pulse wave signal PPG corresponding to each of the first to N-th pressure intervals.

In operation S122, it is determined whether a number of peak values PK of the peak detection signal PPS exceeding at least one threshold is two or more.

Referring to FIG. 17, according to some aspects, the processor 710 determines that a number of peak values PK included in the peak detection signal PPS exceeding at least one threshold value is two or more and analyzes at least one of the first to N-th pressure intervals corresponding to the two or more peak values that exceed the at least one threshold as the regeneration pressure interval P3. In some embodiments, the first to N-th pressure intervals respectively correspond to first to N-th thresholds. In some embodiments, when a peak value PK having a certain magnitude that exceeds a certain threshold is detected in the peak detection signal PPS, the peak detection signal PPS may be unstable and may therefore be unsuitable to be used in calculating a blood pressure of a user. Accordingly, in some embodiments, when the processor 710 determines that the number of peak values PK that exceed the at least one threshold of the peak detection signal PPS is two or more, the processor 710 analyzes pressure intervals corresponding to the peak values PK of the peak detection signal PPS that exceed the at least one threshold as the regeneration pressure intervals P3. For example, referring to FIG. 17, when a first peak value PK1, a second peak value PK2, and a third peak value PK3 that exceed at least one threshold are included in a peak detection signal PPS, the processor 710 may analyze pressure intervals corresponding to the first peak value PK1, the second peak value PK2, and the third peak value PK3 as the regeneration pressure intervals P3.

In operation S123, the processor 710 determines the regeneration pressure interval P3, and the display panel 10 displays the regeneration pressure interval P3 via the first user interface U1.

As described above, when the plurality of peak values PK having the magnitudes exceeding the at least one threshold are detected in the peak detection signal PPS, the peak detection signal PPS may be unstable and may be unsuitable to be used in calculating the blood pressure of the user. Accordingly, in some embodiments, when the number of peak values PK of the peak detection signal PPS that exceed the at least one threshold is two or more, the processor 710 determines the pressure intervals corresponding to the peak values PK of the peak detection signal PPS that exceed the at least one threshold as the regeneration pressure intervals P3.

Referring to FIGS. 18 and 19, according to some aspects, the first area of the display panel 10 displays an image of at least one pressure interval as the regeneration pressure interval P3 via the first user interface U1. For example, in some embodiments, when the processor 710 regenerates the pulse wave signal PPG for any one of the first to N-th pressure intervals, the first user interface U1 displays an image of the regeneration pressure interval P3 on the display panel 10. For example, in some embodiments, when the processor 710 regenerates the K-th pulse wave signal PPG for the K-th pressure interval, the first user interface U1 changes a color of a portion of an image of the pulse wave signal PPG corresponding to the K-th pressure interval into a color different from the rest of the image of the pulse wave signal PPG, or performs shading on a portion of the image of the pulse wave signal PPG corresponding to the K-th pressure interval so that the K-th pressure interval may be identified by the user.

Accordingly, in some embodiments, the first area of the display panel 10 displays an image of the pulse wave signal PPG generated based on the first to N-th pressure intervals via a first user interface U1, and displays an image of at least one pressure interval as the regeneration pressure interval P3 via the first user interface U1.

In operation S124, the pulse wave signal PPG corresponding to the regeneration pressure interval P3 is regenerated.

According to some aspects, the first area of the display panel 10 displays the image of the pulse wave signal PPG regenerated for at least one pressure interval (e.g., the regeneration pressure interval P3). Accordingly, in some embodiments, first area of the display panel 10 displays the image of the pulse wave signal PPG generated for the first to N-th pressure intervals and displays the image of the at least one pressure interval (e.g., the regeneration pressure interval P3).

Accordingly, in some embodiments, when the processor 710 regenerates values of the pulse wave signal PPG for the first to N-th pressure intervals, the regeneration pressure intervals P3 and the regenerated pulse wave signals PPG are displayed on the display panel 10 via the first user interface U1. Accordingly, in some embodiments, the user may identify the regenerated values of the pulse wave signal PPG in real time.

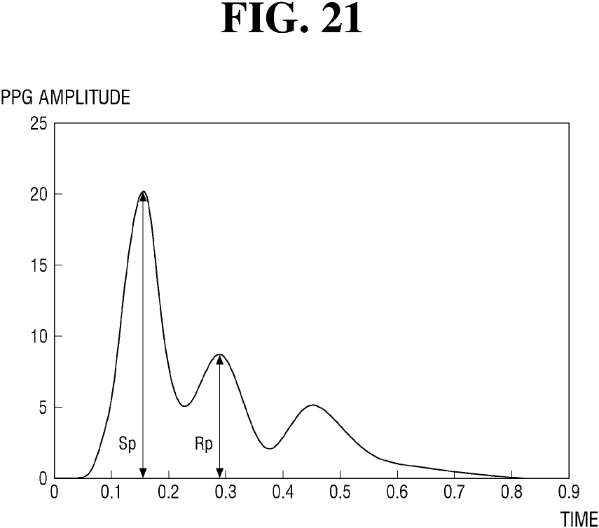

FIG. 20 is a flowchart illustrating a process for regenerating a pulse wave signal according to at least one embodiment. FIGS. 21 to 23 are enlarged graphs of waveforms of the pulse wave signal illustrated in FIG. 16.

Referring to FIG. 20, in operation S131, a reflected pulse wave ratio RI is calculated for each cycle of the pulse wave signal PPG.

Referring to FIGS. 20 and 21, according to some aspects, the processor 710 calculates the reflected pulse wave ratio RI of the pulse wave signal PPG by dividing a wave cycle of the pulse wave signal PPG generated in real time according to a period in which a wave corresponding to a heartbeat and a reflected wave of a blood vessel are sequentially generated. For example, in some embodiments, one cycle of the pulse wave signal PPG includes a plurality of waveforms having different amplitudes. Accordingly, in some embodiments, a peak value PK of a waveform having a greatest amplitude among the plurality of waveforms is a pulse wave maximum value Sp, a peak value PK of a waveform having a second greatest amplitude among the plurality of waveforms is a reflected pulse wave value Rp, and the reflected pulse wave ratio RI is calculated as follows:

$$R I = \frac{R p}{S p} \qquad \text{(1)}$$

In operation S132, it is determined whether there is a pressure interval that corresponds to a reflected pulse wave ratio RI equal to or greater than 1.

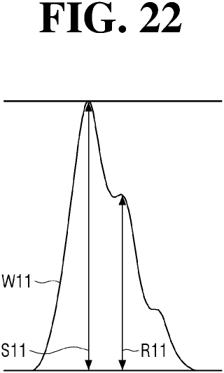

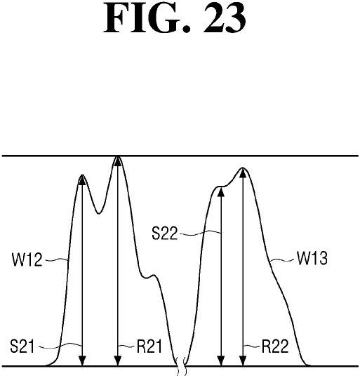

According to some aspects, the processor 710 determines whether there is a pressure interval corresponding to a calculated reflected pulse wave ratio RI equal to or greater than 1. According to some aspects, an ideal pulse wave signal PPG corresponds to a reflected pulse wave ratio RI of less than 1, and an incorrect pulse wave signal PPG corresponds to a reflected pulse wave ratio RI equal to or greater than 1. For example, referring to FIG. 22, a pulse wave maximum value S11 and a reflected pulse wave value R11 of a first pulse wave signal PPG cycle W11 correspond to a pulse wave ratio RI that is less than 1. Accordingly, the first pulse wave signal PPG cycle W11 is ideally detected. In another example, referring to FIG. 23, pulse wave maximum values S21 and S22 and reflected pulse wave values R21 and R22 of a second pulse wave signal PPG cycle W12 and a third pulse wave signal PPG cycle W13 respectively correspond to pulse wave ratios RI that are equal to or greater than 1. Accordingly, the second pulse wave signal PPG cycle W12 and the third pulse wave signal PPG cycle W13 are incorrectly detected.

In operation S133, when there is a pressure interval corresponding to a reflected pulse wave ratio RI equal to or greater than 1 (S132: Y), the processor 710 determines the regeneration pressure interval P3, and the display panel 10 displays a first user interface U1 including the regeneration pressure interval P3.

According to some aspects, when the reflected pulse wave ratio RI corresponding to any one of the first to N-th pressure intervals is equal to or greater than 1, the processor 710 regenerates the pulse wave signal PPG according to an amount of light sensed by the photo-sensor PS when the display device receives a pressure that corresponds to any one pressure interval and according to an optical signal corresponding to the amount of light. For example, referring to FIG. 23, when the reflected pulse wave ratios RI respectively corresponding to the second pulse wave signal cycle W12 and the third pulse wave signal cycle W13 are equal to or greater than 1, the processor 710 determines pressure intervals for the second pulse wave signal cycle W12 and the second pulse wave signal cycle W13 as the regeneration pressure intervals P3. In another example, referring to FIG. 22, when the reflected pulse wave ratio RI corresponding to the first pulse wave signal cycle W11 is less than 1, the processor 710 does not determine a pressure interval for the first pulse wave signal cycle W11 as the regeneration pressure interval P3.

According to some aspects, as described above, the first area of the display panel 10 displays an image of the at least one pressure interval (e.g., the regeneration section P3). A description thereof is substantially the same as that of FIGS. 18 and 19 and is therefore omitted.

According to some aspects, when there is no pressure interval corresponding to a reflected pulse wave ratio RI equal to or greater than 1 (S132: N), the processor 710 does not set the regeneration section P3.

In operation S134, the values of the pulse wave signal PPG corresponding to the regeneration section P3 are regenerated.

Accordingly, in some embodiments, when the processor 710 regenerates the values of the pulse wave signal PPG corresponding to the first to N-th pressure intervals, the regeneration pressure intervals P3 and the regenerated pulse wave signal PPG are displayed on the display panel 10 via the first user interface U1. Accordingly, in some embodiments, the user may identify the regenerated values of the pulse wave signal PPG in real time.

FIG. 24 is a flowchart illustrating a process for calculating a blood pressure of a user using a generated pulse wave signal according to at least one embodiment.

Referring to FIG. 24, in operation ST1, the processor 710 determines whether a peak detection signal PPS may be calculated based on the pulse wave signal PPG.

According to some aspects, the processor 710 generates the peak detection signal PPS using peak values PK of the pulse wave signal PPG. Operation ST1 is an example of or includes aspects of operation S121 described with reference to FIG. 15, and a repeated description thereof is omitted.

In operation ST2, the processor 710 determines whether a pressure value corresponding to a peak value PK of the peak detection signal PPS may be calculated.

According to some aspects, when a peak of the peak detection signal PPS exists, the peak value PK of the peak detection signal PPS exists. Accordingly, in some embodiments, the processor 710 calculates the pressure value corresponding to the peak value PK of the peak detection signal PPS.

In operation ST3, the processor 710 calculates blood pressure information including a systolic blood pressure SBP of the user a diastolic blood pressure DBP of the user, and the like, based on the peak value PK of the peak detection signal PPS).

According to some aspects, the processor 710 calculates the diastolic blood pressure DBP, the systolic blood pressure SBP, and a mean blood pressure according to the pressure value as described with reference to FIG. 13. For example, in some embodiments, the processor 710 calculates a first calculated pressure value PR1 as being equal to about 60% to about 80% of the pressure value. In some embodiments, the processor 710 determines the diastolic blood pressure DBP as the first calculated pressure value PR1. For example, in some embodiments, the processor calculates the diastolic blood pressure as being equal to a value in the range of about 60% to about 80% of the pressure value. According to some aspects, the processor 710 calculates a second calculated pressure value PR2 corresponding to about 120% to about 140% of the pressure value. In some embodiments, the processor 710 determines the systolic blood pressure SBP as the second calculated pressure value PR2. For example, in some embodiments, the processor 710 calculates the systolic blood pressure as being equal to a value in the range of about 120% to about 140% of the pressure value.

According to some aspects, the processor 710 calculates the mean blood pressure based on the diastolic blood pressure DBP and the systolic blood pressure SBP. According to some aspects, the processor 710 calculates the mean blood pressure according to various appropriate formulas and/or algorithms.

For example, in some embodiments, the processor 710 calculates the mean blood pressure MBP according to:

$$M B P = D B P + \frac{1}{3} \left(S B P - D B P\right) \qquad \text{(2)}$$

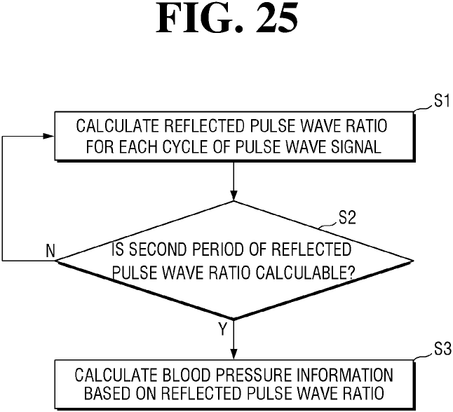

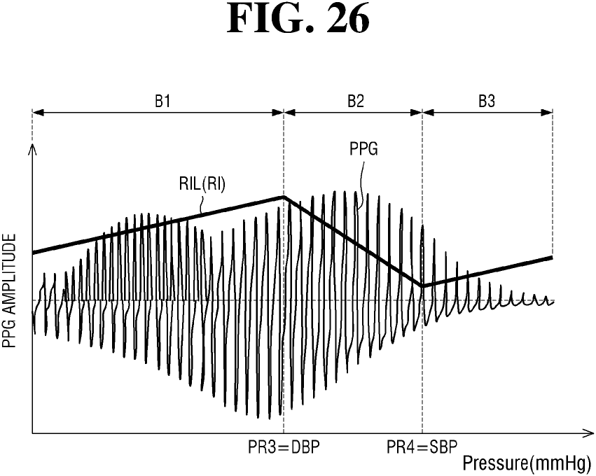

FIG. 25 is a flowchart illustrating a process for calculating a blood pressure using a generated pulse wave signal and a reflected pulse wave ratio according to at least one embodiment. FIG. 26 is a graph illustrating a pulse wave signal according to at least one embodiment.

Referring to FIGS. 25 and 26, in operation S1, a reflected pulse wave ratio RI is calculated for each cycle of the pulse wave signal PPG. Operation S1 is an example of or includes aspects of operation S131 described with reference to FIG. 20, and a repeated description thereof is therefore omitted.

In operation S2, the processor 710 determines whether a second period B2 of the reflected pulse wave ratio RI may be calculated.

Referring to FIG. 26, according to some aspects, the processor 710 sequentially stores detection results of reflected pulse wave ratios RI of reflected pulse waves to pulse wave maximum values, and analyzes the stored reflected pulse wave ratios RI. In this case, as illustrated in FIG. 26, the processor 710 continuously makes changes in magnitude of the reflected pulse wave ratios RI corresponding to the first to N-th pressure intervals to analyze a change in magnitude of reflected pulse wave ratio RI data RIL.

According to some aspects, the reflected pulse wave ratio RI includes a first period B1 in which the reflected pulse wave ratio RI fluctuates within a first range, a second period B2 in which the reflected pulse wave ratio RI fluctuates within a second range, and a third period B3 in which the reflected pulse wave ratio RI fluctuates within a third range. In an example, referring to FIG. 26, the processor 710 analyzes the reflected pulse wave ratio data RIL to analyze a first period B 1 in which the reflected pulse wave ratio RI gently changes within a preset range in a saturated state, a second period B2 in which the reflected pulse wave ratio RI sharply decreases or increases in a preset range within a preset period, a third period B3 in which the reflected pulse wave ratio RI gently changes within a preset range in a saturated state again after it sharply decreases or increases, and the like.

In this example, a width of the first range and a width of the third range is smaller than a width of the second range. In addition, in this example, a gradient of the second period B2 of the reflected pulse wave ratio RI is greater than a gradient of the first period B 1 of the reflected pulse wave ratio RI and a gradient of the third period B3 of the reflected pulse wave ratio RI.

In operation S3, the processor 710 calculates blood pressure information of the user including a systolic blood pressure SBP, a diastolic blood pressure DBP, and the like, based on the reflected pulse wave ratio RI.

According to some aspects, the processor 710 analyzes the reflected pulse wave ratio RI to detect a start point in time of the second period B2. In some embodiments, the processor 710 calculates a third pressure value PR3 corresponding to the pulse wave signal PPG at the start point in time of the second period B2. In some embodiments, the processor 710 determines the diastolic blood pressure DBP as the third pressure value PR3. In some embodiments, the processor 710 analyzes the reflected pulse wave ratio RI to detect a start point in time of the third period B3 after the second period B2. In some embodiments, the processor 710 calculates a fourth pressure value PR4 corresponding to the pulse wave signal PPG at the start point in time of the third period B3. In some embodiments, the processor 710 determines the systolic blood pressure SBP as the fourth pressure value PR4.

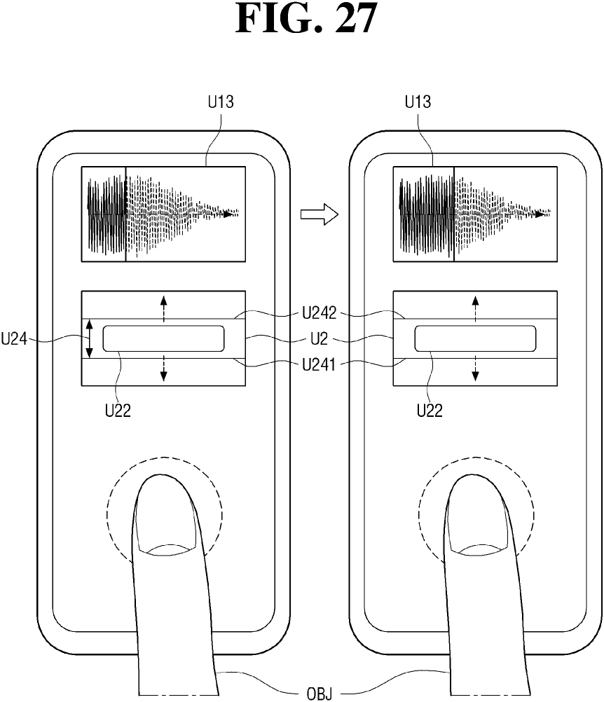

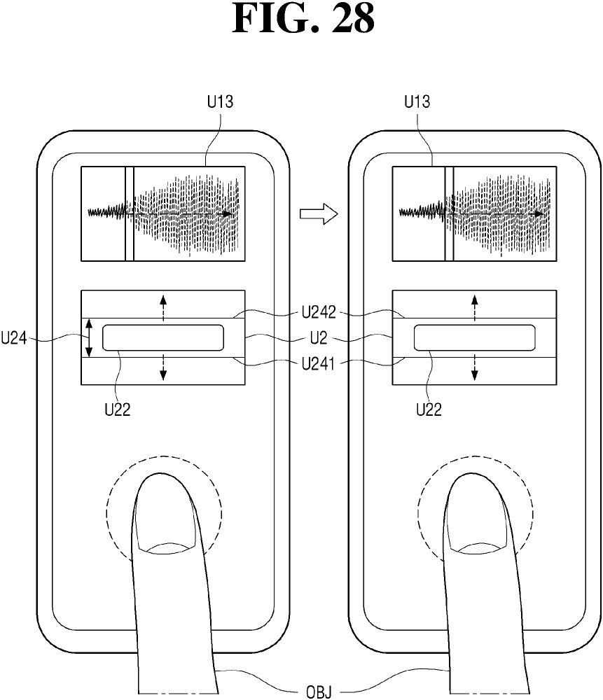

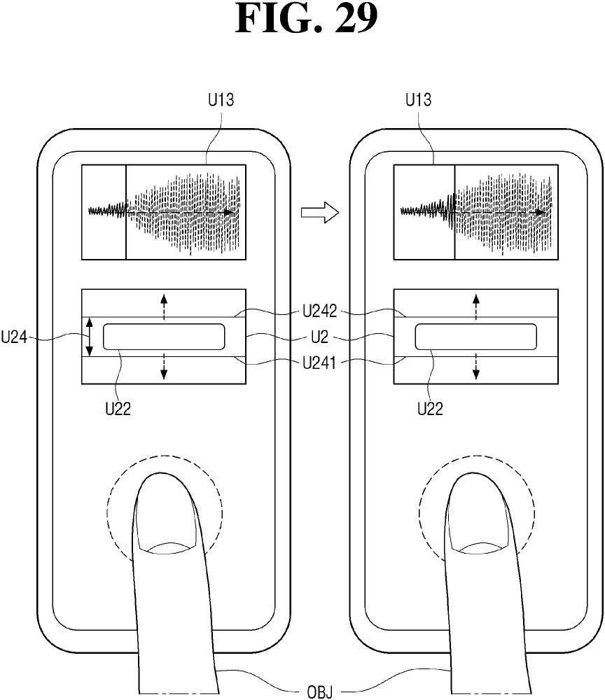

FIG. 27 is a plan view illustrating a user interface according to at least one embodiment. FIGS. 28 and 29 are plan views illustrating user interfaces according to at least one embodiment.

FIGS. 27 to 29 illustrate embodiments that are examples of or include aspects of embodiments described with reference to FIGS. 5 to 24, and a repeated description thereof is omitted. However, FIGS. 27 to 29 illustrate changes made to the first user interface U1 displayed by the display panel 10.

According to some aspects, when the processor 710 generates values of the pulse wave signal PPG based on any one of the first to N-th pressure intervals, a first user interface U13 displays an image of a section of the pulse wave signal PPG that is being generated together with an image of a section of the pulse wave signal PPG that has been generated. In some embodiments, referring to FIG. 27, when the processor 710 generates the K-th pulse wave signal PPG based on the K-th section, the first user interface U13 changes a color of portions of the image of the pulse wave signal PPG corresponding to the first to K-th pressure intervals or performs shading on the portions of the image of the pulse wave signal PPG corresponding to the first to K-th pressure intervals. For example, in some embodiments, portions of the pulse wave signal PPG corresponding to the first to K-th pressure intervals and the subsequent K+1-th to N-th pressure intervals may therefore be separated and identified by the user.

According to some aspects, referring to FIGS. 28 and 29, the pressure request range U24 and the pressure measurement value gradually decrease when generating values of the pulse wave signal PPG based on the first to the N-th pressure intervals.

According to some aspects, when the processor 710 generates the values of the pulse wave signal PPG corresponding to the first to N-th pressure intervals, the display panel 10 displays the images of the values of the generated pulse wave signal PPG via the first user interface U13. Accordingly, the user may identify that the pulse wave signal PPG is generated in real time.

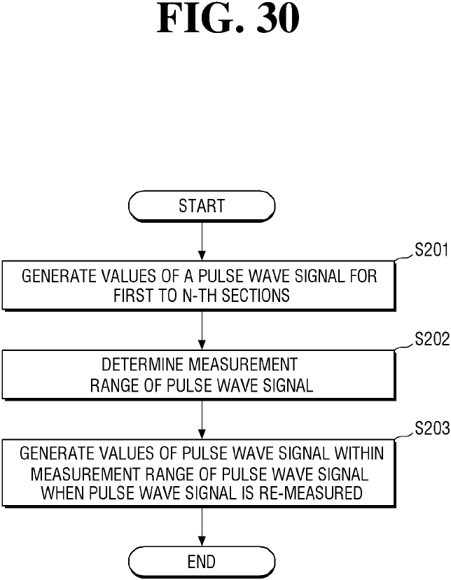

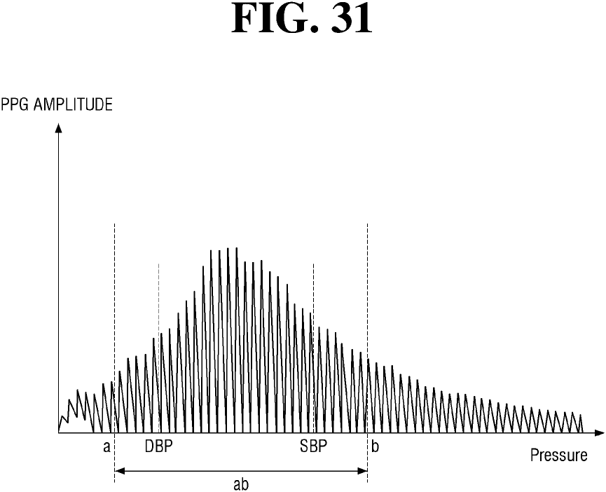

FIG. 30 is a flowchart illustrating a process for blood pressure measurement using a display device according to at least one embodiment. FIG. 31 is a graph illustrating a pulse wave signal according to at least one embodiment.

FIGS. 30 and 31 illustrate embodiments in which values of a pulse wave signal PPG are generated for the first to N-th pressure intervals. Repeated descriptions of aspects of these embodiments provided with reference to FIGS. 5 to 24 are omitted.

Referring to FIG. 30, in operation 201, the processor 710 generates values of a pulse wave signal PPG for the first to N-th pressure intervals. According to some aspects, the processor 710 calculates blood pressure information including the diastolic blood pressure DBP and the systolic blood pressure SBP as described with reference to FIGS. 5 to 24.

In operation S202, the processor 710 determines a measurement range of the pulse wave signal PPG. Referring to FIG. 31, for example, when the systolic blood pressure SBP and the diastolic blood pressure DBP are calculated, the processor 710 determines a measurement range of the pulse wave signal PPG (as bounded by pressure values “a” and “b”, for example) including the systolic blood pressure SBP and the diastolic blood pressure DBP.

In operation S203, the processor 710 re-measures the pulse wave signal PPG and generates values of the pulse wave signal PPG within the measurement range of the pulse wave signal PPG. For example, in some embodiments, the processor 710 generates the pulse wave signal PPG having values within the measurement range of the pulse wave signal PPG. According to some aspects, the processor 710 generates only values of a pulse wave signal PPG corresponding to a pressure interval in a process of calculating the blood pressure based on the pulse wave signal PPG. Accordingly, in some embodiments, a time for measuring the blood pressure of the user using the display device is shortened.

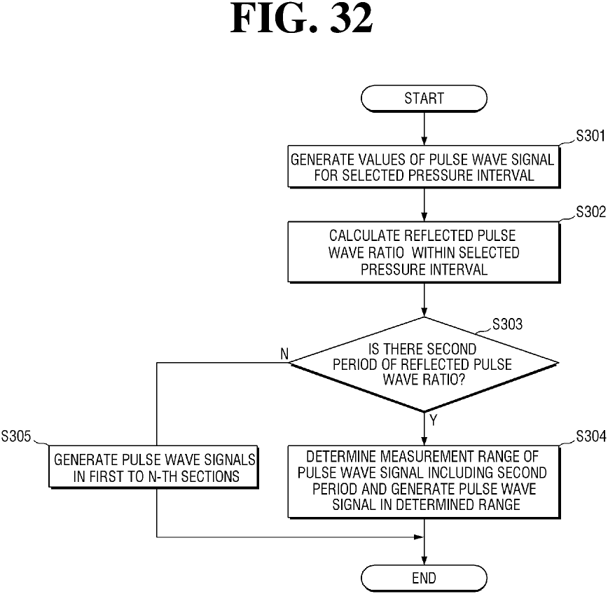

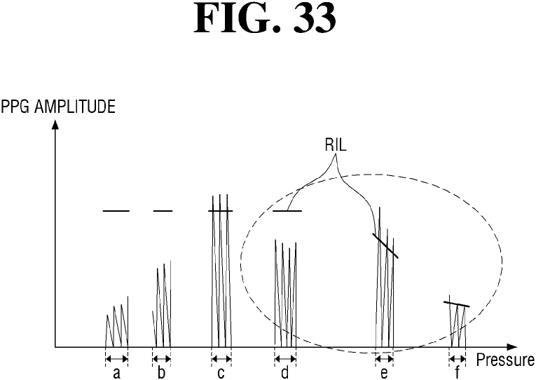

FIG. 32 is a flowchart illustrating a blood pressure measurement process using a display device according to at least one embodiment. FIGS. 33 to 35 are graphs illustrating pulse wave signals according to at least one embodiment.

FIGS. 32 to 35 illustrate embodiments in which values of a pulse wave signal PPG are generated for the first to N-th pressure intervals. Repeated descriptions of aspects of these embodiments provided with reference to FIGS. 5 to 24 are omitted.

Referring to FIG. 32, in operation S301, the processor 710 generates a pulse wave signal PPG for a pressure interval.

Referring to FIG. 33, in some embodiments, the processor 710 generates values of the pulse wave signal PPG for a selected pressure interval of the first to N-th pressure intervals. For example, the processor 710 may select a pressure interval a, b, c, d, e, f, etc. as the selected pressure interval, and may generate values of the pulse wave signal PPG corresponding to the selected pressure interval.

In operation S302, the processor 710 calculates a reflected pulse wave ratio RI corresponding to the values of the pulse wave signal PPG corresponding to the selected pressure interval.

According to some aspects, the processor 710 calculates the reflected pulse wave ratio RI and calculates reflected pulse wave ratio data RIL according to the reflected pulse wave ratio RI. A process for calculating the second period B2 of the reflected pulse wave ratio data RIL by the processor 710 is described with reference to FIGS. 5 to 24.

In operation S303, the processor 710 determines whether the second period B2 is calculated in the reflected pulse wave ratio RI.

In operation S304, when the second period B2 is calculated (S303: Y), the processor 710 measures the blood pressure again, the processor 710 generates values of a pulse wave signal PPG in a measurement range of the pulse wave signal PPG including the second period B2.

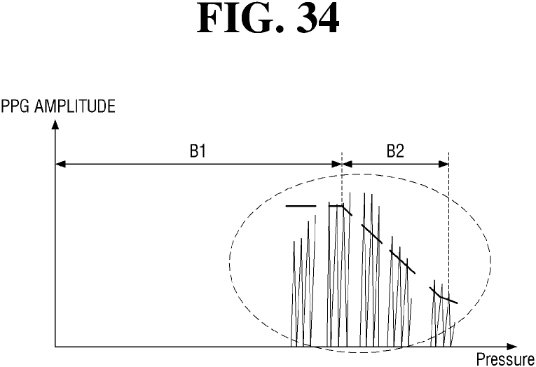

Referring to FIG. 34, when the processor 710 measures the blood pressure again, the processor 710 generates values of the pulse wave signal PPG within a pressure range including a pressure corresponding to the second period B2 of the reflected pulse wave ratio RI. In some embodiments, the processor 710 generates only values of a pulse wave signal PPG corresponding to the selected pressure interval in a process of calculating the blood pressure based on the pulse wave signal PPG.

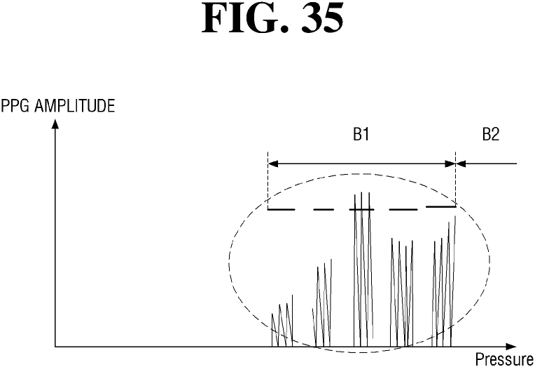

Referring to FIG. 35, when the second period B2 is not calculated (S303: N), the processor 710 measures the blood pressure again and generates values of the pulse wave signal PPG corresponding to the first to N-th pressure intervals (S305).

According to some aspects, the processor 710 only generates values of the pulse wave signal PPG corresponding to the selected section in a process of calculating the blood pressure based on the pulse wave signal PPG. Accordingly, in some embodiments, a time required for measuring the blood pressure using the display device is shortened.

While the inventive concept has been particularly shown and described with reference to embodiments thereof, it will be understood that various changes in form and details may be made therein without departing from the spirit and scope of the present disclosure.

What is claimed is:

1. A display device comprising:
a display panel including a pixel configured to display an image and a photo-sensor configured to sense incident light;
a pressure sensor disposed on one surface of the display panel and configured to sense a pressure applied via a portion of a user’s body; and
a processor configured to:
generate a pulse wave signal according to an amount of incident light sensed by the photo-sensor in response to determining that a pressure measurement value corresponding to the pressure is within a pressure request range corresponding to a pressure interval, and
calculate blood pressure information based on the pulse wave signal.

2. The display device of claim 1, wherein the processor is further configured to:
analyze the pressure interval as a regeneration section; and
regenerate the pulse wave signal according to an amount of light sensed by the photo-sensor corresponding to the pressure interval.

3. The display device of claim 2, wherein the processor is further configured to:
generate a peak detection signal using peak values of the pulse wave signal; and
determine the pressure interval as the regeneration section when a number of peak values of the peak detection signal that exceed at least one threshold is two or more.

4. The display device of claim 3, wherein a first area of the display panel is configured to:
display an image of values of the pulse wave signal generated corresponding to a plurality of pressure intervals via a first user interface; and
display an image of the regeneration section via the first user interface.

5. The display device of claim 1, wherein a first area of the display panel is configured to display a first image of values of the pulse wave signal generated based on the pressure interval via a first user interface, and a second area of the display panel is configured to display a second image of the pressure request range and the pressure measurement value via a second user interface.

6. The display device of claim 5,
wherein the pressure request range includes a first requested pressure and a second requested pressure higher than the first requested pressure, and the second image further includes the first requested pressure and the second requested pressure.

7. The display device of claim 1,
wherein the processor is further configured to:
generate a peak detection signal using a peak value of the pulse wave signal;
calculate a pressure value corresponding to the peak value; and
calculate a diastolic blood pressure, a systolic blood pressure, and a mean blood pressure according to the pressure value.

8. The display device of claim 7,
wherein the processor is further configured to:
calculate the diastolic blood pressure as being equal to a value in a range of about 60% to about 80% of the pressure value; and
calculate the systolic blood pressure as being equal to a value in a range of about 120% to about 140% of the pressure value.

9. The display device of claim 1,
wherein a greatest amplitude in a cycle of the pulse wave signal is a pulse wave maximum value, a second greatest amplitude in the cycle of the pulse wave signal is a reflected pulse wave value, and the processor is further configured to calculate a reflected pulse wave ratio as a ratio of the reflected pulse wave value to the pulse wave maximum value.

10. The display device of claim 9,
wherein the reflected pulse wave ratio includes a first period in which the reflected pulse wave ratio fluctuates within a first range, a second period in which the reflected pulse wave ratio fluctuates within a second range, and a third period in which the reflected pulse wave ratio fluctuates within a third range, and a width of the first range and a width of the third range are smaller than a width of the second range.

11. The display device of claim 10,
wherein the processor is further configured to:
analyze the reflected pulse wave ratio to detect a start point in time of the second period;
calculate a third pressure value corresponding to the pulse wave signal at the start point in time of the second period;
determine a diastolic blood pressure as the third pressure value;
calculate a fourth pressure value corresponding to the pulse wave signal at a start point in time of the third period after the second period; and
determine a systolic blood pressure as the fourth pressure value.

12. The display device of claim 9,
wherein the reflected pulse wave ratio is equal to or greater than one, and the processor is further configured to regenerate the pulse wave signal according to a second amount of light sensed by the photo-sensor.

13. A method for measuring blood pressure, the method comprising:
sensing, via a pressure sensor of a display device, a pressure applied via a portion of a user’s body;
generating a pulse wave signal according to an amount of light sensed by a photo-sensor of a display device in response to determining that a pressure measurement value corresponding to the pressure is within a pressure request range corresponding to a pressure interval;
regenerating the pulse wave signal according to a second amount of light sensed by the photo-sensor in response to analyzing the pressure interval as a regeneration pressure interval; and
calculating blood pressure information based on the regenerated pulse wave signal.

14. The method of claim 13, further comprising:
generating a peak detection signal using peak values of the pulse wave signal; and
determining the pressure interval as the regeneration pressure interval when the number of peak values of the peak detection signal that exceed a threshold is two or more.

15. The method of claim 13, further comprising:
displaying an image of the pulse wave signal via a first user interface; and
displaying an image of the pressure measurement value and a pressure request range corresponding to the pressure measurement value via a second user interface.

16. The method of claim 15,
further comprising displaying an image of the regeneration pressure interval via the first user interface.

17. The method of claim 1,
wherein a greatest amplitude in a cycle of the pulse wave signal is a pulse wave maximum value, a second greatest amplitude in the cycle of the pulse wave signal is a reflected pulse wave value, and further comprising calculating a reflected pulse wave ratio as the ratio of the reflected pulse wave value to the pulse wave maximum value.

18. The method of claim 17,
further comprising determining that the pulse wave ratio is equal to or greater than 1, wherein the pulse wave signal is regenerated based on the determination.

19. The method of claim 1, further comprising:
generating a peak detection signal using peak values of the pulse wave signal;
calculating a pressure value corresponding to the peak value of the peak detection signal; and
calculating a diastolic blood pressure, a systolic blood pressure, and a mean blood pressure according to the pressure value.

20. A method for measuring blood pressure, comprising:
displaying a pressure request range via a user interface;
receiving a pressure within the pressure request range via a portion of a user’s body;
generating a pulse wave signal based on the pressure and a first amount of light reflected from the user;
determining that two or more values of the pulse wave signal exceed a threshold;
regenerating the pulse wave signal to obtain a regenerated pulse wave signal and a second amount of light reflected from the user; and
calculating the blood pressure of the user based on the regenerated pulse wave signal.
